SEQUENCE LISTING (4 sequences)

SEQ ID NO: 1 - protein, 450 aa, Artificial Sequence
  Synthetic Sequence
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 211 aa, Artificial Sequence
  Synthetic Sequence
EIVLTQSPAIMSASPGERVTMTCSASSGVNYMHWYQQKPGTSPRRWIYDTSKLASGVPARFSGSGSGTDYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 115 aa, Artificial Sequence
  Synthetic Sequence
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSS

SEQ ID NO: 4 - protein, 108 aa, Artificial Sequence
  Synthetic Sequence
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKR

(19) United States
(12) Patent Application Publication
(10) Pub. No.: US 2015/0306242 A1
(43) Pub. Date: Oct. 29, 2015

(54) PROCESS FOR PREPARING STABLE ANTIBODY MAYTANSINOID CONJUGATES

(71) Applicant: IMMUNOGEN, INC., Waltham, MA (US)

(72) Inventors: Xinfang Li, Newton, MA (US); Jared M. Worful, Pittsburgh, PA (US)

(21) Appl. No.: 14/430,834

(22) PCT Filed: Oct. 4, 2013
(86) PCT No.: PCT/US13/63415

§ 371 Date: Mar. 24, 2015

Related U.S. Application Data
(60) Provisional application No. 61/709,914, filed on Oct. 4, 2012.

(51) Int. Cl. A61K 47/48; C07K 16/32; C07K 16/30; A61K 31/537; C07K 16/28

(52) U.S. Cl. CPC A61K 47/48369; A61K 47/48384; A61K 47/48715; A61K 31/537; C07K 16/2803; A61K 47/48561; C07K 16/3092; A61K 47/48569; C07K 16/2896; C07K 16/28; C07K 16/2863; C07K 16/2878; C07K 16/32; A61K 47/48246

(57) ABSTRACT

The invention provides processes for manufacturing cell-binding agent-cytotoxic agent conjugates of improved stability comprising performing the modification reaction at a high pH. The inventive processes comprise contacting a cell-binding agent with a bifunctional crosslinking reagent in a solution having a pH of 7.1 to 9 to covalently attach a linker to the cell-binding agent and thereby prepare a mixture comprising cell-binding agents having linkers bound thereto.

### CROSS-REFERENCE TO RELATED APPLICATIONS

This patent application claims the benefit of U.S. Provisional Patent Application No. 61/709,914, filed Oct. 4, 2012, which is incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 8,233 Byte ASCII (Text) file named “714289SequenceListing.TXT,” created on Oct. 3, 2013.

### BACKGROUND OF THE INVENTION

Antibody-Drug-Conjugates (ADC's), which are useful for the treatment of cancer and other diseases, are commonly composed of three distinct elements: a cell-binding agent; a linker; and a cytotoxic agent. Commonly used manufacturing processes comprise a modification step, in which the cell-binding agent is reacted with a bifunctional linker at a pH of 7.0 or below to form a cell-binding agent covalently attached to a linker having a reactive group, and a conjugation step, in which the modified cell-binding agent is reacted with a cytotoxic agent to form a covalent chemical bond from the linker (using the reactive group) to the cytotoxic agent.

Despite advances in preparing antibody-drug conjugates, current processes are limited by several factors. For example, the binding of a bifunctional cross-linking agent to an antibody is heterogeneous under the conditions currently employed in the art, resulting in a conjugate comprising stable amide bonds and unstable ester bonds. It is thought that the presence of unstable ester bonds in the conjugate lead to the slow release of the drug from the conjugate and conjugate instability.

Recent clinical trials have shown a promising role for antibody-drug conjugates in the treatment of many different types of cancers. Thus, there remains a need for improved processes of preparing antibody-drug conjugates that are more stable and are of higher purity than antibody-drug conjugates produced by current processes. The invention provides such a process. These and other advantages of the invention, as well as additional inventive features, will be apparent from the description of the invention provided herein.

### BRIEF SUMMARY OF THE INVENTION

The invention provides a process for preparing a cell-binding agent having a linker bound thereto, which process comprises contacting a cell-binding agent with a bifunctional crosslinking reagent in a solution having a pH of 7.1 to 9 (preferably 7.5 to 9) to covalently attach a linker to the cell-binding agent and thereby prepare a mixture comprising cell-binding agents having linkers bound thereto.

In one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent in a solution having a pH of 7.1 to 9 (preferably 7.5 to 9) to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and (d) subjecting the second mixture to tangential flow filtration, selective precipitation, non-adsorptive chromatography, adsorptive filtration, adsorptive chromatography, or a combination thereof to purify the cell-binding agent chemically coupled through the linker to the cytotoxic agent from the other components of the second mixture and thereby prepare a purified second mixture of cell-binding agent chemically coupled through the linker to the cytotoxic agent.

Another embodiment of the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent in a solution having a pH of 7.1 to 9 (preferably 7.5 to 9) to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and (c) subjecting the second mixture to tangential flow filtration, selective precipitation, non-adsorptive chromatography, adsorptive filtration, adsorptive chromatography, or a combination thereof, to purify the cell binding agent chemically coupled through the linker to the cytotoxic agent from the other components of the second mixture and thereby prepare a purified second mixture of cell binding agent chemically coupled through the linker to the cytotoxic agent.

The invention also provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent-linker compound comprising a cytotoxic agent chemically coupled to a linker in a solution having a pH of 7.1 to 9 (preferably 7.5 to 9) to covalently attach the cytotoxic agent-linker compound to the cell-binding agent and thereby prepare a mixture comprising the cell-binding agent-cytotoxic agent conjugate (b) subjecting the mixture comprising the cell-binding agent-cytotoxic agent conjugate to tangential flow filtration, selective precipitation, non-adsorptive chromatography, adsorptive filtration, adsorptive chromatography or a combination thereof to purify the conjugate.

The present invention also includes a conjugate comprising a cell-binding agent chemically coupled to a cytotoxic agent prepared according to the processes described herein.

### BRIEF DESCRIPTION OF THE SEVERAL VIEWS OF THE DRAWING(S)

FIG. 1 is a line graph depicting the percent free maytansinoid (FM %) (y-axis) present in huN901-SPP-DM1 conjugates over time (x-axis) when stored at 4° C. The conjugates were prepared by performing the modification reaction at pH 7.0, pH 7.5, pH 7.8, or pH 8.0.

FIG. 2 is a line graph depicting the percent free maytansinoid (FM %) (y-axis) present in huN901-SMCC-DM1 conjugates over time (x-axis) when stored at 4° C. The conjugates were prepared by performing the modification reaction at pH 6.7, pH 7, or pH 7.5.

FIG. 3 is a line graph depicting the percent free maytansinoid (FM %) (y-axis) present in humanized anti-CD37 antibody-SPDB-DM4 conjugates over time (x-axis) when stored at 25° C. The conjugates were prepared by a process comprising either one purification step (one purif.) or two purification steps (two purif.) wherein the modification reaction was performed at pH 6.5 or pH 7.5.

FIG. 4 is a line graph depicting the percent free maytansinoid (FM %) (y-axis) present in humanized anti-CD37 antibody-SPDB-DM4 conjugates over time (x-axis) when stored at 4° C. The conjugates were prepared by a process comprising either one purification step (one purif.) or two purification steps (two purif.) wherein the modification reaction was performed at pH 6.5 or pH 7.5.

FIG. 5 is a line graph depicting the percent free maytansinoid (FM %) (y-axis) present in humanized anti-CD37 antibody-SPDB-DM4 conjugates over time (x-axis) when stored at 40° C. The conjugates were prepared by a process comprising either one purification step (one purif.) or two purification steps (two purif.) wherein the modification reaction was performed at pH 6.5 or pH 7.5.

FIG. 6 is a line graph depicting the percent free maytansinoid (FM %) (y-axis) present in humanized anti-CD33 antibody-SPDB-DM4 conjugates over time (x-axis) when stored at 4° C. The conjugates were prepared by a process comprising either one purification step (one purif.) or two purification steps (two purif.) wherein the modification reaction was performed at pH 6.5 or pH 7.5.

FIG. 7 is a line graph depicting the percent free maytansinoid (FM %) (y-axis) present in humanized anti-CD33 antibody-SPDB-DM4 conjugates over time (x-axis) when stored at 25° C. The conjugates were prepared by a process comprising either one purification step (one purif.) or two purification steps (two purif.) wherein the modification reaction was performed at pH 6.5 or pH 7.5.

FIG. 8 is a line graph depicting the percent free maytansinoid (FM %) (y-axis) present in humanized anti-CD33 antibody-SPDB-DM4 conjugates over time (x-axis) when stored at 40° C. The conjugates were prepared by a process comprising either one purification step (one purif.) or two purification steps (two purif.) wherein the modification reaction was performed at pH 6.5 or pH 7.5.

### DESCRIPTION OF THE INVENTION

One of ordinary skill in the art will appreciate that conjugates comprising a cell-binding agent, such as an antibody, chemically coupled to a cytotoxic agent (“antibody-cytotoxic agent conjugates”) typically are prepared by modifying an antibody with a bifunctional crosslinking reagent at a low pH (i.e., pH 7.0 or below), purifying the antibody having linkers bound thereto, conjugating a cytotoxic agent to the antibody having linkers bound thereto, and purifying the antibody-cytotoxic agent conjugate. The invention improves upon such methods by maximizing the amount of linker stably bound to the cell-binding agent and minimizing undesirable side reactions that lead to conjugate instability. In particular, it was surprisingly discovered that performing the modification reaction (reaction of the cell-binding agent with the linker) at a high pH (e.g., a pH of 7.5 or higher), increases the level of desirable species of cell-binding agents having a linker stably bound thereto and reduces the level of undesirable reaction products (e.g., cell-binding agents having a linker unstably bound thereto), thereby making the process suitable for large scale manufacturing. Accordingly, the invention provides processes for manufacturing cell-binding agent-cytotoxic agent conjugates of increased stability comprising performing the modification reaction at a high pH.

The invention provides a process for preparing a cell-binding agent having a linker bound thereto, which process comprises contacting a cell-binding agent with a bifunctional crosslinking reagent in a solution having a pH of 7.1 to 9 (preferably 7.5 to 9) to covalently attach a linker to the cell-binding agent and thereby prepare a mixture comprising cell-binding agents having linkers bound thereto. For example, the inventive process comprises contacting a cell-binding agent with a bifunctional crosslinking reagent in a solution having a pH of about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In one embodiment, the inventive process comprises contacting a cell-binding agent with a bifunctional crosslinking reagent in a solution having a pH of about 7.5 to about 9, about 7.5 to about 8.5, about 7.5 to about 8, about 7.5 to about 7.8, about 7.8 to about 9, about 7.8 to about 8.5, about 7.8 to about 8, about 8 to about 9, about 8 to about 8.5, or about 8.5 to about 9. In one embodiment, the inventive process comprises contacting a cell-binding agent with a bifunctional crosslinking reagent in a solution having a pH of about 7.5 (e.g., a pH of 7.3 to 7.7 or a pH of 7.4 to 7.6). In another embodiment, the inventive process comprises contacting a cell-binding agent with a bifunctional crosslinking reagent in a solution having a pH of about 7.8 (e.g., a pH of 7.6 to 8.0 or a pH of 7.7 to 7.9). In another embodiment, the inventive process comprises contacting a cell-binding agent with a bifunctional crosslinking reagent in a solution having a pH of about 8 (e.g., a pH of 7.8 to 8.2 or a pH of 7.9 to 8.1). In another embodiment, the inventive process comprises contacting a cell-binding agent with a bifunctional crosslinking reagent in a solution having a pH of about 8.5 (e.g., a pH of 8.3 to 8.7 or a pH of 8.4 to 8.6). In another embodiment, the inventive process comprises contacting a cell-binding agent with a bifunctional crosslinking reagent in a solution having a pH of about 9.0 (e.g., a pH of 8.8 to 9.2 or a pH of 8.9 to 9.1).

In another embodiment, the inventive process comprises contacting a cell-binding agent with a bifunctional crosslinking reagent in a solution having a pH that is greater than 7.5 (e.g., a pH of 7.6 to about 9, 7.7 to about 9, about 7.8 to about 9, about 7.9 to about 9, 7.6 to about 8.5, 7.6 to about 8, 7.7 to about 8.5, 7.7 to about 8, about 7.8 to about 8.4, about 7.8 to about 8.2, about 8 to about 9, or about 8 to about 8.5). For example, the inventive process comprises contacting a cell-binding agent with a bifunctional crosslinking reagent in a solution having a pH of 7.6, 7.7, 7.8, 7.9, 8, 8.1, 8.2, 8.3, 8.4, 8.5, 8.6, 8.7, 8.8, 8.9, or 9.

In another embodiment, the inventive process comprises contacting a cell-binding agent with a bifunctional crosslinking reagent in a solution having a pH that is greater than or equal to 7.5 (e.g., a pH of 7.5 to about 9, 7.6 to about 9, 7.7 to about 9, about 7.8 to about 9, about 7.9 to about 9, 7.5 to about 8.5, 7.6 to about 8.5, 7.5 to about 8, 7.6 to about 8, 7.7 to about 8.5, 7.7 to about 8, about 7.8 to about 8.4, about 7.8 to about 8.2, about 8 to about 9, or about 8 to about 8.5). For example, the inventive process comprises contacting a cell-binding agent with a bifunctional crosslinking reagent in a solution having a pH of 7.5, 7.6, 7.7, 7.8, 7.9, 8, 8.1, 8.2, 8.3, 8.4, 8.5, 8.6, 8.7, 8.8, 8.9, or 9.

The inventive process comprises performing the modification reaction (reaction of the cell-binding agent with the linker) at a temperature of 16° C. or higher. For example, reaction of the cell-binding agent with the linker can occur at room temperature (e.g., about 20° C. to about 30° C. or about 20° C. to about 25° C.), or at an elevated temperature (e.g., about 30° C. to about 37° C.). In one embodiment, reaction of the cell-binding agent with the linker occurs at a temperature of 16° C. to about 37° C. (e.g., 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C.), 16° C. to about 30° C., or about 20° C. to about 37° C. In one embodiment, reaction of the cell-binding agent with the linker occurs at a temperature of 16° C. to about 25° C. In a preferred embodiment, reaction of the cell-binding agent with the linker occurs at room temperature (e.g., about 20° C. to about 30° C. or about 20° C. to about 25° C.), and preferably at a temperature of about 20° C.

Any suitable buffering agent can be used. Suitable buffering agents include, for example, a citrate buffer, an acetate buffer, a succinate buffer, and a phosphate buffer. In a preferred embodiment, the buffering agent is selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and combinations thereof.

In one embodiment, the inventive process comprises contacting a cell-binding agent with a bifunctional crosslinking reagent in a solution having a high pH (e.g., a pH of 7.5 or greater) at room temperature (e.g., about 20° C.). In a preferred embodiment, the inventive process comprises contacting a cell-binding agent with a bifunctional crosslinking reagent in a solution having a pH about 7.8 at a temperature of about 20° C. In another preferred embodiment, the inventive process comprises contacting a cell-binding agent with a bifunctional crosslinking reagent in a solution having a pH about 8 at a temperature of about 20° C. In another preferred embodiment, the inventive process comprises contacting a cell-binding agent with a bifunctional crosslinking reagent in a solution having a pH about 8.5 at a temperature of about 20° C.

In accordance with the inventive method, contacting a cell-binding agent with a bifunctional crosslinking reagent produces a first mixture comprising the cell-binding agent having linkers bound thereto, as well as reactants and other by-products. In some embodiments of the invention, the first mixture comprises the cell-binding agent having linkers stably and unstably bound thereto, as well as reactants and other by-products. A linker is “stably” bound to the cell-binding agent when the covalent bond between the linker and the cell-binding agent is not substantially weakened or severed under normal storage conditions over a period of time, which could range from a few months to a few years. In contrast, a linker is “unstably” bound to the cell-binding agent when the covalent bond between the linker and the cell-binding agent is substantially weakened or severed under normal storage conditions over a period of time, which could range from a few months to a few years.

In one embodiment of the invention, purification of the modified cell-binding agent from reactants and by-products is carried out by subjecting the first mixture to a purification process. In this regard, the first mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. This first purification step provides a purified first mixture, i.e., an increased concentration of the cell-binding agents having linkers bound thereto and a decreased amount of unbound bifunctional crosslinking reagent, as compared to the first mixture prior to purification in accordance with the invention. Preferably, the first mixture is purified using tangential flow filtration.

After purification of the first mixture to obtain a purified first mixture of cell-binding agents having linkers bound thereto, a cytotoxic agent is conjugated to the cell-binding agents having linkers bound thereto in the first purified mixture by reacting the cell-binding agents having linkers bound thereto with a cytotoxic agent in a solution having a pH from about 4 to about 9, wherein a second mixture comprising (i) the cell-binding agent chemically coupled through the linker to the cytotoxic agent, (ii) free cytotoxic agent, and (iii) reaction by-products is produced.

Optionally, purification of the modified cell-binding agent may be omitted. Thus, in one embodiment of the invention, the first mixture comprising the cell-binding agent having linkers bound thereto, as well as reactants and other by-products, is not subjected to a purification process. In such a situation, the cytotoxic agent may be added simultaneously with the crosslinking reagent or at some later point, e.g., 1, 2, 3, or more hours after addition of the crosslinking reagent to the cell-binding agent. The modified cell-binding agent is conjugated to a cytotoxic agent (e.g., a maytansinoid) by reacting the modified cell-binding agent with the cytotoxic agent in a solution having a pH from about 4 to about 9, wherein the conjugation step results in formation of a mixture of stable cell-binding agent-cytotoxic agent conjugates, non-stable cell-binding agent-cytotoxic agent conjugates, non-conjugated cytotoxic agent (i.e., “free” cytotoxic agent), reactants, and by-products.

As used herein, the term “free cytotoxic agent” refers to any form of the cytotoxic agent that is not chemically coupled to the cell-binding agent through the linker (i.e., the “free cytotoxic agent” can include the cytotoxic agent alone represented by D, the cytotoxic agent coupled with the linker or a linker derivative (e.g., hydrolyzed derivative) represented by D-L, and cytotoxic agent dimers represented by D-D and D-L-D, wherein D represents the cytotoxic agent and L represents the bifunctional crosslinking reagent or derivative thereof. In contrast, the cell-binding agent cytotoxic agent conjugate can be represented by CBA-L-D, wherein CBA represents the cell-binding agent).

The conjugation reaction preferably is performed at a pH of about 4 to about pH 9 (e.g., a pH of about 4.5 to about 8.5, about 5 to about 8, about 5.5 to about 7.5, or about 6.0 to about 7). In some embodiments, the conjugation reaction is performed at a pH of about 6 to about 6.5 (e.g., a pH of 5.5 to 7, a pH of 5.7 to 6.8, a pH of 5.8 to 6.7, a pH of 5.9 to 6.6, or a pH of 6 to 6.5), a pH of about 6 or below (e.g., a pH of about 4 to 6, about 4 to about 5.5, about 5 to 6) or at a pH of about 6.5 or greater (e.g., a pH of 6.5 to about 9, about 7 to about 9, about 7.5 to about 9, or 6.5 to about 8). In one embodiment, the conjugation reaction is performed at a pH of about 4 to a pH less than 6 or at a pH of greater than 6.5 to 9. When the conjugation step is performed at a pH of about 6.5 or greater, some sulfhydryl-containing cytotoxic agents may be prone to dimerize by disulfide-bond formation. In one embodiment, removal of trace metals and/or oxygen from the reaction mixture, as well as optional addition of antioxidants or the use of linkers with more reactive leaving groups, or addition of cytotoxic agent in more than one aliquot, may be required to allow for efficient reaction in such a situation.

The inventive process may optionally include the addition of sucrose to the conjugation step used in the inventive process to increase solubility and recovery of the cell-binding agent-cytotoxic agent conjugates. Desirably, sucrose is added at a concentration of about 0.1% (wv) to about 20% (wv) (e.g., about 0.1% (wv), 1% (wv), 5% (wv), 10% (wv), 15% (wv), or 20% (wv)). Preferably, sucrose is added at a concentration of about 1% (wv) to about 10% (wv) (e.g., about 0.5% (wv), about 1% (wv), about 1.5% (wv), about 2% (wv), about 3% (wv), about 4% (wv), about 5% (wv), about 6% (wv), about 7% (wv), about 8% (wv), about 9% (wv), about 10% (wv), or about 11% (wv)). In addition, the conjugation reaction also can comprise the addition of a buffering agent. Any suitable buffering agent known in the art can be used. Suitable buffering agents include, for example, a citrate buffer, an acetate buffer, a succinate buffer, and a phosphate buffer. In a preferred embodiment, the buffering agent is selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and combinations thereof.

Following the conjugation step, the conjugate is subjected to a purification step. In this regard, the conjugation mixture can be purified using tangential flow filtration (TFF), e.g., a membrane-based tangential flow filtration process, non-adsorptive chromatography, adsorptive chromatography, adsorptive filtration, or selective precipitation, or any other suitable purification process, as well as combinations thereof. One of ordinary skill in the art will appreciate that purification after the conjugation step enables the isolation of a stable conjugate comprising the cell-binding agent chemically coupled to the cytotoxic agent.

In one embodiment, the invention provides a process for preparing a conjugate comprising a cell-binding agent chemically coupled to a cytotoxic agent, which process comprises a first purification step after the modification step and a second purification step after the conjugation step. For example, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent in a solution having a pH of 7.1 to 9 (preferably 7.5 to 9) to covalently attach a linker to the cell-binding agent and thereby prepare a first mixture comprising cell-binding agents having linkers bound thereto, (b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto, (c) conjugating a cytotoxic agent to the cell-binding agents having linkers bound thereto in the purified first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and (d) subjecting the second mixture to tangential flow filtration, selective precipitation, non-adsorptive chromatography, adsorptive filtration, adsorptive chromatography, or a combination thereof to purify the cell-binding agent chemically coupled through the linker to the cytotoxic agent from the other components of the second mixture and thereby prepare a purified second mixture of cell-binding agent chemically coupled through the linker to the cytotoxic agent.

In one embodiment of the invention, tangential flow filtration (TFF, also known as cross flow filtration, ultrafiltration and diafiltration) and/or adsorptive chromatography resins are utilized in the purification steps. For example, the inventive process can comprise a first purification step using TFF after the modification step and a second purification step using TFF after the conjugation step. Alternatively, the inventive process can comprise a first purification step using adsorptive chromatography after the modification step and a second purification step using adsorptive chromatography after the conjugation step. The inventive process also can comprise a first purification step using adsorptive chromatography after the modification step and a second purification step using TFF after the conjugation step or a first purification step using TFF after the modification step and a second purification step using adsorptive chromatography after the conjugation step. In one embodiment, the adsorptive chromatography is an ion-exchange chromatography, e.g., ceramic hydroxyapatite (CHT) chromatography.

In one embodiment of the invention, non-adsorptive chromatography is utilized as the purification step. For example, the inventive process can comprise a first purification step using non-adsorptive chromatography after the modification step and a second purification step using non-adsorptive chromatography after the conjugation step.

In another embodiment, the invention provides a process for preparing a conjugate comprising a cell-binding agent chemically coupled to a cytotoxic agent, which process comprises a single purification step after the conjugation step. For example, the invention provides a process for preparing a cell-binding agent-cytotoxic agent conjugate comprising a cell-binding agent chemically coupled to a cytotoxic agent through a linker, which process comprises (a) contacting a cell-binding agent with a bifunctional crosslinking reagent in a solution having a pH of 7.1 to 9 (preferably 7.5 to 9) to covalently attach a linker to the cell-binding agent and thereby prepare a first mixture comprising cell-binding agents having linkers bound thereto, (b) conjugating a cytotoxic agent to the cell-binding agents having linkers bound thereto in the first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and (c) subjecting the second mixture to tangential flow filtration, selective precipitation, non-adsorptive chromatography, adsorptive filtration, adsorptive chromatography, or a combination thereof, to purify the cell binding agent chemically coupled through the linker to the cytotoxic agent from the other components of the second mixture and thereby prepare a purified second mixture of cell binding agent chemically coupled through the linker to the cytotoxic agent, wherein the first mixture comprising the cell-binding agent having linkers bound thereto (as well as reactants and other by-products) prepared in step (a) is not subjected to a purification process prior to step (b).

In one embodiment, the invention provides a process for preparing a conjugate comprising a cell-binding agent chemically coupled to a cytotoxic agent, wherein the process comprises conjugating a pre-formed cytotoxic-agent-linker compound to a cell-binding agent, as described in U.S. Pat. No. 6,441,163 and U.S. Patent Application Publication Nos. 2011/0003969 and 2008/0145374. For example, in one embodiment, the invention provides a process for preparing a cell-binding agent-cytotoxic agent conjugate comprising a cell-binding agent chemically coupled to a cytotoxic agent through a linker, which process comprises (a) contacting a cell-binding agent with a cytotoxic agent-linker compound comprising a cytotoxic agent chemically coupled to a linker in a solution having a pH of 7.5 to 9 to covalently attach the cytotoxic agent-linker compound to the cell-binding agent and thereby prepare a mixture comprising the cell-binding agent-cytotoxic agent conjugate (b) subjecting the mixture comprising the cell-binding agent-cytotoxic agent conjugate to tangential flow filtration, selective precipitation, non-adsorptive chromatography, adsorptive filtration, adsorptive chromatography or a combination thereof to purify the conjugate.

In one embodiment, the cytotoxic agent-linker compound is prepared by contacting a cytotoxic agent with a bifunctional crosslinking reagent comprising a linker to covalently attach the cytotoxic agent to the linker. The cytotoxic agent-linker compound optionally is subjected to purification before contacting cytotoxic agent-linker compound with the cell-binding agent. Thus, in one embodiment, the cytotoxic agent-linker compound is purified before contacting the cell-binding agent. In another embodiment, the cytotoxic agent-linker compound is not purified before contacting the cell-binding agent.

In one embodiment of the invention, the inventive process comprises two separate purification steps to purify the cell-binding agent-cytotoxic agent conjugate. Any combination of purification methods described herein can be used to purify the cell-binding agent-cytotoxic agent conjugate. In a preferred embodiment, tangential flow filtration and adsorptive chromatography are utilized as the two separate purification steps to purify the cell-binding agent-cytotoxic agent conjugate (e.g., adsorptive chromatography (e.g., ion-exchange chromatography, preferably CHT chromatography) followed by tangential flow filtration).

In one embodiment, the inventive process described herein further comprises formulating the purified cell-binding agent-cytotoxic agent conjugate into a formulation buffer using tangential flow filtration.

Any suitable TFF systems may be utilized for the purification steps described herein, including a Pellicon type system (Millipore, Billerica, Mass.), a Sartocon Cassette system (Sartorius A G, Edgewood, N.Y.), and a Centrasette type system (Pall Corp., East Hills, N.Y.).

Any suitable adsorptive chromatography resin may be utilized for the purification steps described herein. Preferred adsorptive chromatography resins include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof. Examples of suitable hydroxyapatite resins include ceramic hydroxyapatite (CHT Type I and Type II, Bio-Rad Laboratories, Hercules, Calif.), HA Ultrogel hydroxyapatite (Pall Corp., East Hills, N.Y.), and ceramic fluoroapatite (CFT Type I and Type II, Bio-Rad Laboratories, Hercules, Calif.). An example of a suitable HCIC resin is MEP Hypercel resin (Pall Corp., East Hills, N.Y.). Examples of suitable HIC resins include Butyl-Sepharose, Hexyl-Sepaharose, Phenyl-Sepharose, and Octyl Sepharose resins (all from GE Healthcare, Piscataway, N.J.), as well as Macro-prep Methyl and Macro-Prep t-Butyl resins (Biorad Laboratories, Hercules, Calif.). Examples of suitable ion exchange resins include SP-Sepharose, CM-Sepharose, and Q-Sepharose resins (all from GE Healthcare, Piscataway, N.J.), and Unosphere S resin (Bio-Rad Laboratories, Hercules, Calif.). Examples of suitable mixed mode ion exchangers include Bakerbond ABx resin (JT Baker, Phillipsburg N.J.). Examples of suitable IMAC resins include Chelating Sepharose resin (GE Healthcare, Piscataway, N.J.) and Profinity IMAC resin (Bio-Rad Laboratories, Hercules, Calif.). Examples of suitable dye ligand resins include Blue Sepharose resin (GE Healthcare, Piscataway, N.J.) and Affi-gel Blue resin (Bio-Rad Laboratories, Hercules, Calif.). Examples of suitable affinity resins include Protein A Sepharose resin (e.g., MabSelect, GE Healthcare, Piscataway, N.J.), where the cell-binding agent is an antibody, and lectin affinity resins, e.g. Lentil Lectin Sepharose resin (GE Healthcare, Piscataway, N.J.), where the cell-binding agent bears appropriate lectin binding sites. Alternatively an antibody specific to the cell-binding agent may be used. Such an antibody can be immobilized to, for instance, Sepharose 4 Fast Flow resin (GE Healthcare, Piscataway, N.J.). Examples of suitable reversed phase resins include C4, C8, and C18 resins (Grace Vydac, Hesperia, Calif.).

Any suitable non-adsorptive chromatography resin may be utilized for the purification steps described herein. Examples of suitable non-adsorptive chromatography resins include, but are not limited to, SEPHADEX™ G-25, G-50, G-100, SEPHACRYL™ resins (e.g., S-200 and S-300), SUPERDEX™ resins (e.g., SUPERDEX™ 75 and SUPERDEX™ 200), BIO-GEL® resins (e.g., P-6, P-10, P-30, P-60, and P-100), and others known to those of ordinary skill in the art.

In one embodiment, the inventive process further comprises a holding step to release the unstably bound linkers from the cell-binding agent. The holding step comprises holding the mixture after modification of the cell-binding agent with a bifunctional crosslinking reagent, after conjugation of a cytotoxic agent to the cell-binding agents having linkers bound thereto, and/or after a purification step.

The holding step comprises maintaining the solution at a suitable temperature (e.g., about 2° C. to about 37° C.) for a suitable period of time (e.g., about 1 hour to about 1 week) to release the unstably bound linkers from the cell-binding agent while not substantially releasing the stably bound linkers from the cell-binding agent. In one embodiment, the holding step comprises maintaining the solution at a low temperature (e.g., about 2° C. to about 10° C. or about 4° C.), at room temperature (e.g., about 20° C. to about 30° C. or about 20° C. to about 25° C.), or at an elevated temperature (e.g., about 30° C. to about 37° C.).

The duration of the holding step depends on the temperature at which the holding step is performed. For example, the duration of the holding step can be substantially reduced by performing the holding step at elevated temperature, with the maximum temperature limited by the stability of the cell-binding agent-cytotoxic agent conjugate. The holding step can comprise maintaining the solution for about 1 hour to about 1 day (e.g., about 1 hour, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, or about 24 hours), about 5 hours to about 1 week, about 12 hours to about 1 week (e.g., about 12 hours, about 16 hours, about 20 hours, about 24 hours, about 2 days, about 3 days, about 4 days, about 5 days, about 6 days, or about 7 days), for about 12 hours to about 1 week (e.g., about 12 hours, about 16 hours, about 20 hours, about 24 hours, about 2 days, about 3 days, about 4 days, about 5 days, about 6 days, or about 7 days), or about 1 day to about 1 week.

In one embodiment, the holding step comprises maintaining the solution at a temperature of about 2° C. to about 8° C. for a period of at least about 12 hours for up to 1 day.

The pH value for the holding step preferably is about 4 to about 10. In one embodiment, the pH value for the holding step is about 4 or more, but less than about 6 (e.g., 4 to 5.9) or about 5 or more, but less than about 6 (e.g., 5 to 5.9). In another embodiment, the pH values for the holding step range from about 6 to about 10 (e.g., about 6.5 to about 9, about 6 to about 8). For example, pH values for the holding step can be about 6, about 6.5, about 7, about 7.5, about 8, about 8.5, about 9, about 9.5, or about 10.

The holding step can be performed before or after the cell-binding agent is conjugated to the cytotoxic agent. In one embodiment, the holding step is performed directly after the modification of the cell-binding agent with the bifunctional crosslinking reagent. For example, the inventive process comprises a holding step after modification of the cell-binding agent with a bifunctional crosslinking reagent and before conjugation. After modification of the cell-binding agent, a purification step may be performed before the hold step and/or after the hold step, but prior to the conjugation step. In another embodiment, the holding step is performed directly after conjugation of the cytotoxic agent to the cell-binding agent having linkers bound thereto and prior to purification step. In another embodiment, the holding step is performed after the conjugation and purification steps and followed by an additional purification step.

In specific embodiments, the holding step can comprise incubating the mixture at 4° C. at a pH of about 6-7.5 for about 12 hours to about 1 week, incubating the mixture at 25° C. at a pH of about 6-7.5 for about 12 hours to about 1 week, incubating the mixture at 4° C. at a pH of about 4.5-5.9 for about 5 hours to about 5 days, or incubating the mixture at 25° C. at a pH of about 4.5-5.9 for about 5 hours to about 1 day.

The invention provides a process for preparing compositions of stable conjugates comprising a cell-binding agent chemically coupled to a cytotoxic agent, wherein the compositions are substantially free of unstable conjugates. In this respect, the invention provides a process for preparing cell-binding agent-cytotoxic agent conjugate of substantially high purity and stability. Such compositions can be used for treating diseases because of the high purity and stability of the conjugates. Compositions comprising a cell-binding agent, such as an antibody, chemically coupled to a cytotoxic agent, such as a maytansinoid, are described in, for example, U.S. Pat. No. 7,374,762. In one aspect of the invention, a cell-binding agent-cytotoxic agent conjugate of substantially high purity has one or more of the following features: (a) greater than about 90% (e.g., greater than or equal to about 91%, 92%, 93%, 94%, 95%, 96%, 97%, 98%, 99%, or 100%), preferably greater than about 95%, of conjugate species are monomeric, (b) unconjugated linker level in the conjugate preparation is less than about 10% (e.g., less than or equal to about 9%, 8%, 7%, 6%, 5%, 4%, 3%, 2%, 1%, or 0%) (relative to total linker), (c) less than 10% of conjugate species are crosslinked (e.g., less than or equal to about 9%, 8%, 7%, 6%, 5%, 4%, 3%, 2%, 1%, or 0%), (d) free cytotoxic agent level in the conjugate preparation is less than about 2% (e.g., less than or equal to about 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (relative to total cytotoxic agent), and/or (e) no substantial increase in free cytotoxic agent level upon storage (e.g., after about 1 week, about 2 weeks, about 3 weeks, about 1 month, about 2 months, about 3 months, about 4 months, about 5 months, about 6 months, about 1 year, about 2 years, or about 5 years). “Substantial increase” in free cytotoxic agent level means that after certain storage time, the increase in the level of free cytotoxic agent is less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.2%, about 2.5%, about 2.7%, about 3.0%, about 3.2%, about 3.5%, about 3.7%, or about 4.0%.

As used herein, the term “unconjugated linker” refers to the cell-binding agent that is covalently linked with the bifunctional crosslinking reagent, wherein the cell-binding agent is not covalently coupled to the cytotoxic agent through the linker of the bifunctional crosslinking reagent (i.e., the “unconjugated linker” can be represented by CBA-L, wherein CBA represents the cell-binding agent and L represents the bifunctional crosslinking reagent. In contrast, the cell-binding agent cytotoxic agent conjugate can be represented by CBA-L-D, wherein D represents the cytotoxic agent).

In one embodiment, the average molar ratio of the cytotoxic agent to the cell-binding agent in the cell-binding agent cytotoxic agent conjugate is about 1 to about 10, about 2 to about 7, about 3 to about 5, about 2.5 to about 4.5 (e.g., about 2.5, about 2.6, about 2.7, about 2.8, about 2.9, about 3.0, about 3.1, about 3.3, about 3.4, about 3.5, about 3.6, about 3.7, about 3.8, about 3.9, about 4.0, about 4.1, about 4.2, about 4.3, about 4.4, about 4.5), about 3.0 to about 4.0, about 3.2 to about 4.2, about 4.5 to 5.5 (e.g., about 4.5, about 4.6, about 4.7, about 4.8, about 4.9, about 5.0, about 5.1, about 5.2, about 5.3, about 5.4, about 5.5).

The cell-binding agent can be any suitable agent that binds to a cell, typically and preferably an animal cell (e.g., a human cell). The cell-binding agent preferably is a peptide or a polypeptide or a glycotope. Suitable cell-binding agents include, for example, antibodies (e.g., monoclonal antibodies and fragments thereof), interferons (e.g. alpha, beta, gamma), lymphokines (e.g., IL-2, IL-3, IL-4, IL-6), hormones (e.g., insulin, TRH (thyrotropin releasing hormone), MSH (melanocyte-stimulating hormone), steroid hormones, such as androgens and estrogens), growth factors and colony-stimulating factors such as EGF, TGF-alpha, FGF, VEGF, G-CSF, M-CSF and GM-CSF (Burgess, Immunology Today 5:155-158 (1984)), nutrient-transport molecules (e.g., transferrin), vitamins (e.g., folate) and any other agent or molecule that specifically binds a target molecule on the surface of a cell.

Where the cell-binding agent is an antibody, it binds to an antigen that is a polypeptide and may be a transmembrane molecule (e.g., receptor) or a ligand, such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-13; platelet-derived growth factor (PDGF); fibroblast growth factor (FGF) such as aFGF and bFGF; fibroblast growth factor receptor such as FGFR24 and FGFR3, epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins, EpCAM, GD3, FLT3, PSMA, PSCA, MUC1, MUC16, STEAP, CEA, TENB2, EphA receptors, EphB receptors, folate receptor, FOLRl, mesothelin, cripto, alphaᵥbeta₆, integrins, VEGF, VEGFR, EGFR, transferrin receptor, IRTA1, IRTA2, IRTA3, IRTA4, IRTA5; CD proteins such as CD2, CD3, CD4, CD5, CD6, CD8, CD11, CD14, CD19, CD20, CD21, CD22, CD25, CD26, CD28, CD30, CD33, CD36, CD37, CD38, CD40, CD44, CD52, CD55, CD56, CD59, CD70, CD79, CD80. CD81, CD103, CD105, CD134, CD137, CD138, CD152 or an antibody which binds to one or more tumor-associated antigens or cell-surface receptors disclosed in U.S. Patent Application Publication No. 2008/0171040 or U.S. Patent Application Publication No. 2008/0305044 and are incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CD11a, CD11b, CD11c, CD18, an ICAM, VLA-4 and VCAM; a tumor associated antigen such as HER2, HER3 or HER4 receptor; endoglin, c-Met, IGF1R, prostate antigens such as PCA3, PSA, PSGR, NGEP, PSMA, PSCA, TMEFF2, and STEAP1; LGR5, B7H4, and fragments of any of the above-listed polypeptides.

Additionally, GM-CSF, which binds to myeloid cells can be used as a cell-binding agent to diseased cells from acute myelogenous leukemia. IL-2 which binds to activated T-cells can be used for prevention of transplant graft rejection, for therapy and prevention of graft-versus-host disease, and for treatment of acute T-cell leukemia. MSH, which binds to melanocytes, can be used for the treatment of melanoma, as can antibodies directed towards melanomas. Folic acid can be used to target the folate receptor expressed on ovarian and other tumors. Epidermal growth factor can be used to target squamous cancers such as lung and head and neck. Somatostatin can be used to target neuroblastomas and other tumor types.

Cancers of the breast and testes can be successfully targeted with estrogen (or estrogen analogues) or androgen (or androgen analogues) respectively as cell-binding agents

The term “antibody,” as used herein, refers to any immunoglobulin, any immunoglobulin fragment, such as Fab, Fab′, F(ab′)₂, dsFv, sFv, minibodies, diabodies, tribodies, tetrabodies (Parham, J. Immunol. 131: 2895-2902 (1983); Spring et al. J. Immunol. 113: 470-478 (1974); Nisonoff et al. Arch. Biochem. Biophys. 89: 230-244 (1960), Kim et al., Mol. Cancer Ther., 7: 2486-2497 (2008), Carter, Nature Revs., 6: 343-357 (2006)), or immunoglobulin chimera, which can bind to an antigen on the surface of a cell (e.g., which contains a complementarity determining region (CDR)). Any suitable antibody can be used as the cell-binding agent. One of ordinary skill in the art will appreciate that the selection of an appropriate antibody will depend upon the cell population to be targeted. In this regard, the type and number of cell surface molecules (i.e., antigens) that are selectively expressed in a particular cell population (typically and preferably a diseased cell population) will govern the selection of an appropriate antibody for use in the inventive composition. Cell surface expression profiles are known for a wide variety of cell types, including tumor cell types, or, if unknown, can be determined using routine molecular biology and histochemistry techniques.

The antibody can be polyclonal or monoclonal, but is most preferably a monoclonal antibody. As used herein, “polyclonal” antibodies refer to heterogeneous populations of antibody molecules, typically contained in the sera of immunized animals. “Monoclonal” antibodies refer to homogenous populations of antibody molecules that are specific to a particular antigen. Monoclonal antibodies are typically produced by a single clone of B lymphocytes (“B cells”). Monoclonal antibodies may be obtained using a variety of techniques known to those skilled in the art, including standard hybridoma technology (see, e.g., Khler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). In brief, the hybridoma method of producing monoclonal antibodies typically involves injecting any suitable animal, typically and preferably a mouse, with an antigen (i.e., an “immunogen”). The animal is subsequently sacrificed, and B cells isolated from its spleen are fused with human myeloma cells. A hybrid cell is produced (i.e., a “hybridoma”), which proliferates indefinitely and continuously secretes high titers of an antibody with the desired specificity in vitro. Any appropriate method known in the art can be used to identify hybridoma cells that produce an antibody with the desired specificity. Such methods include, for example, enzyme-linked immunosorbent assay (ELISA), Western blot analysis, and radioimmunoassay. The population of hybridomas is screened to isolate individual clones, each of which secretes a single antibody species to the antigen. Because each hybridoma is a clone derived from fusion with a single B cell, all the antibody molecules it produces are identical in structure, including their antigen binding site and isotype. Monoclonal antibodies also may be generated using other suitable techniques including EBV-hybridoma technology (see, e.g., Haskard and Archer, J. Immunol. Methods, 74(2): 361-67 (1984), and Roder et al., Methods Enzymol., 121: 140-67 (1986)), bacteriophage vector expression systems (see, e.g., Huse et al., Science, 246: 1275-81 (1989)), or phage display libraries comprising antibody fragments, such as Fab and scFv (single chain variable region) (see, e.g., U.S. Pat. Nos. 5,885,793 and 5,969,108, and International Patent Application Publications WO 92/01047 and WO 99/06587).

The monoclonal antibody can be isolated from or produced in any suitable animal, but is preferably produced in a mammal, more preferably a mouse or human, and most preferably a human. Methods for producing an antibody in mice are well known to those skilled in the art and are described herein. With respect to human antibodies, one of ordinary skill in the art will appreciate that polyclonal antibodies can be isolated from the sera of human subjects vaccinated or immunized with an appropriate antigen. Alternatively, human antibodies can be generated by adapting known techniques for producing human antibodies in non-human animals such as mice (see, e.g., U.S. Pat. Nos. 5,545,806, 5,569,825, and 5,714,352, and U.S. Patent Application Publication No. 2002/0197266 A1).

While being the ideal choice for therapeutic applications in humans, human antibodies, particularly human monoclonal antibodies, typically are more difficult to generate than mouse monoclonal antibodies. Mouse monoclonal antibodies, however, induce a rapid host antibody response when administered to humans, which can reduce the therapeutic or diagnostic potential of the antibody-cytotoxic agent conjugate. To circumvent these complications, a monoclonal antibody preferably is not recognized as “foreign” by the human immune system.

To this end, phage display can be used to generate the antibody. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete human antibody is reconstituted comprising the selected variable domain. Nucleic acid sequences encoding the reconstituted antibody are introduced into a suitable cell line, such as a myeloma cell used for hybridoma production, such that human antibodies having the characteristics of monoclonal antibodies are secreted by the cell (see, e.g., Janeway et al., supra, Huse et al., supra, and U.S. Pat. No. 6,265,150). Alternatively, monoclonal antibodies can be generated from mice that are transgenic for specific human heavy and light chain immunoglobulin genes. Such methods are known in the art and described in, for example, U.S. Pat. Nos. 5,545,806 and 5,569,825, and Janeway et al., supra.

Most preferably the antibody is a humanized antibody. As used herein, a “humanized” antibody is one in which the complementarity-determining regions (CDR) of a mouse monoclonal antibody, which form the antigen binding loops of the antibody, are grafted onto the framework of a human antibody molecule. Owing to the similarity of the frameworks of mouse and human antibodies, it is generally accepted in the art that this approach produces a monoclonal antibody that is antigenically identical to a human antibody but binds the same antigen as the mouse monoclonal antibody from which the CDR sequences were derived. Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235: 959-973 (1994). While the antibody employed in the conjugate of the inventive composition most preferably is a humanized monoclonal antibody, a human monoclonal antibody and a mouse monoclonal antibody, as described above, are also within the scope of the invention.

Antibody fragments that have at least one antigen binding site, and thus recognize and bind to at least one antigen or receptor present on the surface of a target cell, also are within the scope of the invention. In this respect, proteolytic cleavage of an intact antibody molecule can produce a variety of antibody fragments that retain the ability to recognize and bind antigens. For example, limited digestion of an antibody molecule with the protease papain typically produces three fragments, two of which are identical and are referred to as the Fab fragments, as they retain the antigen binding activity of the parent antibody molecule. Cleavage of an antibody molecule with the enzyme pepsin normally produces two antibody fragments, one of which retains both antigen-binding arms of the antibody molecule, and is thus referred to as the F(ab′)₂ fragment. Reduction of a F(ab′)₂ fragment with dithiothreitol or mercaptoethylamine produces a fragment referred to as a Fab′ fragment. A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7: 697-704 (1994)). Antibody fragments in the context of the invention, however, are not limited to these exemplary types of antibody fragments. Any suitable antibody fragment that recognizes and binds to a desired cell surface receptor or antigen can be employed. Antibody fragments are further described in, for example, Parham, J. Immunol., 131: 2895-2902 (1983), Spring et al., J. Immunol., 113: 470-478 (1974), and Nisonoff et al., Arch. Biochem. Biophys., 89: 230-244 (1960). Antibody-antigen binding can be assayed using any suitable method known in the art, such as, for example, radioimmunoassay (RIA), ELISA, Western blot, immunoprecipitation, and competitive inhibition assays (see, e.g., Janeway et al., supra, and U.S. Patent Application Publication No. 2002/0197266 A1).

In addition, the antibody can be a chimeric antibody or an antigen binding fragment thereof. By “chimeric” it is meant that the antibody comprises at least two immunoglobulins, or fragments thereof, obtained or derived from at least two different species (e.g., two different immunoglobulins, such as a human immunoglobulin constant region combined with a murine immunoglobulin variable region). The antibody also can be a domain antibody (dAb) or an antigen binding fragment thereof, such as, for example, a camelid antibody (see, e.g., Desmyter et al., Nature Struct. Biol., 3: 752, (1996)), or a shark antibody, such as, for example, a new antigen receptor (IgNAR) (see, e.g., Greenberg et al., Nature, 374: 168 (1995), and Stanfield et al., Science, 305: 1770-1773 (2004)).

Any suitable antibody can be used in the context of the invention. For example, the monoclonal antibody J5 is a murine IgG2a antibody that is specific for Common Acute Lymphoblastic Leukemia Antigen (CALLA) (Ritz et al., Nature, 283: 583-585 (1980)), and can be used to target cells that express CALLA (e.g., acute lymphoblastic leukemia cells). The monoclonal antibody MY9 is a murine IgG1 antibody that binds specifically to the CD33 antigen (Griffin et al., Leukemia Res., 8: 521 (1984)), and can be used to target cells that express CD33 (e.g., acute myelogenous leukemia (AML) cells).

Similarly, the monoclonal antibody anti-B4 (also referred to as B4) is a murine IgG1 antibody that binds to the CD19 antigen on B cells (Nadler et al., J. Immunol., 131: 244-250 (1983)), and can be used to target B cells or diseased cells that express CD19 (e.g., non-Hodgkin's lymphoma cells and chronic lymphoblastic leukemia cells). N901 is a murine monoclonal antibody that binds to the CD56 (neural cell adhesion molecule) antigen found on cells of neuroendocrine origin, including small cell lung tumor, which can be used in the conjugate to target drugs to cells of neuroendocrine origin. The J5, MY9, and B4 antibodies preferably are resurfaced or humanized prior to their use as part of the conjugate. Resurfacing or humanization of antibodies is described in, for example, Roguska et al., Proc. Natl. Acad. Sci. USA, 91: 969-73 (1994). In one embodiment, the anti-B4 antibody is huB4. In another embodiment, the anti-B4 antibody comprises a heavy chain and a light chain, wherein the heavy chain has the following sequence

(SEQ ID NO: 1)
QVQLVQPGAE VVKPGASVKL SCKTSGYTFT SNWMHWVKQA
PGQGLEWIGE IDPSDSYTNY NQNFQGKAKL TVDKSTSTAY
MEVSSLRSDD TAVYYCARGS NPYYYAMDYW GQGTSVTVSS
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPGK

and the light chain has the following sequence

(SEQ ID NO: 2)
EIVLTQSPAI MSASPGERVT MTCSASSGVN YMHWYQQKPG
TSPRRWIYDT SKLASGVPAR FSGSGSGTDY SLTISSMEPE
DAATYYCHQR GSYTFGGGTK LEIKRTVAAP SVFIFPPSDE
QLKSGTASVV CLLNNFYPRE AKVQWKVDNA LQSGNSQESV
TEQDSKDSTY SLSSTLTLSK ADYEKHKVYA CEVTHQGLSS
PVTKSFNRGE C.

In addition, the monoclonal antibody C242 binds to the CanAg antigen (see, e.g., U.S. Pat. No. 5,552,293), and can be used to target the conjugate to CanAg expressing tumors, such as colorectal, pancreatic, non-small cell lung, and gastric cancers. HuC242 is a humanized form of the monoclonal antibody C242 (see, e.g., U.S. Pat. No. 5,552,293). The hybridoma from which HuC242 is produced is deposited with ECACC identification Number 90012601. HuC242 can be prepared using CDR-grafting methodology (see, e.g., U.S. Pat. Nos. 5,585,089, 5,693,761, and 5,693,762) or resurfacing technology (see, e.g., U.S. Pat. No. 5,639,641). HuC242 can be used to target the conjugate to tumor cells expressing the CanAg antigen, such as, for example, colorectal, pancreatic, non-small cell lung, and gastric cancer cells.

To target ovarian cancer and prostate cancer cells, an anti-MUC1 antibody can be used as the cell-binding agent in the conjugate. Anti-MUC1 antibodies include, for example, anti-HMFG-2 (see, e.g., Taylor-Papadimitriou et al., Int. J. Cancer, 28: 17-21 (1981)), hCTMO1 (see, e.g., van Hof et al., Cancer Res., 56: 5179-5185 (1996)), and DS6. Prostate cancer cells also can be targeted with the conjugate by using an anti-prostate-specific membrane antigen (PSMA) as the cell-binding agent, such as J591 (see, e.g., Liu et al., Cancer Res., 57: 3629-3634 (1997)). Moreover, cancer cells that express the Her2 antigen, such as breast, prostate, and ovarian cancers, can be targeted with the conjugate by using anti-HER2 antibodies, e.g., trastuzumab, as the cell-binding agent. Cells that express epidermal growth factor receptor (EGFR) and variants thereof, such as the type III deletion mutant, EGFRvIII, can be targeted with the conjugate by using anti-EGFR antibodies. Anti-EGFR antibodies are described in International Patent Application Nos. PCT/US11/058,385 and PCT/US11/058,378. Anti-EGFRvIII antibodies are described in U.S. Pat. Nos. 7,736,644 and 7,628,986, and U.S. Patent Application Publications 2010/0111979; 2009/0240038; 2009/0175887; 2009/0156790; and 2009/0155282. Anti-IGF-IR antibodies that bind to insulin-like growth factor receptor, such as those described in U.S. Pat. No. 7,982,024, also can be used in the conjugate. Antibodies that bind to CD27L, Cripto, CD138, CD38, EphA2, integrins, CD37, folate, CD20, PSGR, NGEP, PSCA, TMEFF2, STEAP1, endoglin, and Her3 also can be used in the conjugate.

In one embodiment, the antibody is selected from the group consisting of huN901, anti-CD33 antibody (e.g., huMy9-6), huB4, huC242, an anti-HER2 antibody (e.g., trastuzumab), bivatuzumab, sibrotuzumab, rituximab, huDS6, anti-mesothelin antibodies described in International Patent Application Publication WO 2010/124797 (such as MF-T), anti-cripto antibodies described in U.S. Patent Application Publication 2010/0093980 (such as huB3F6), anti-CD138 antibodies described in U.S. Patent Application Publication 2007/0183971 (such as B-B4 or humanized B-B4 or nBT062), anti-EGFR antibodies described in International Patent Application Publications WO 2012/058592 and WO 2012/058588 (such as EGFR-7), anti-EGFRvIII antibodies described U.S. Pat. Nos. 7,736,644 and 7,628,986 and U.S. Patent Application Publications 2010/0111979, 2009/0240038, 2009/0175887, 2009/0156790 and 2009/0155282, humanized EphA2 antibodies described in International Patent Application Publications WO 2011/039721 and WO 2011/039724 (such as 2H11R35R74); anti-CD38 antibodies described in International Patent Application Publication WO 2008/047242 (such as hu38SB19), anti-folate receptor antibodies described in International Patent Application Publication WO 2011/106528, and U.S. Patent Application Publication 2012/0009181 (e.g., huMov19 version 1.0 or 1.6); anti-IGF1R antibodies described in U.S. Pat. Nos. 5,958,872, 6,596,743, and 7,982,024; anti-CD37 antibodies described in U.S. Patent Application Publication 2011/0256153 (e.g., huCD37-3 version 1.0); anti-integrin αᵥβ₆ antibodies described in U.S. Patent Application Publication 2006/0127407 (e.g., CNTO95); and anti-Her3 antibodies described in International Patent Application Publication WO 2012/019024. In one embodiment of the invention, the antibody is not huN901, or CNTO95. In one embodiment, the anti-CD37 antibody is huCD37-3, wherein the antibody comprises a variable heavy chain and a variable light chain, wherein the variable heavy chain has the following sequence

(SEQ ID NO: 3)
QVQVQESGPGLVAPSQTLSITCTVSGFSLTTSGVSWVRQPPGKGLEWLGV
IWGDGSTNYHPSLKSRLSIKKDHSKSQVFLKLNSLTAADTATYYCAKGGY
SLAHWGQGTLVTVSS

and the variable light chain has the following sequence

(SEQ ID NO: 4)
DIQMTQSPSSLSVSVGERVTITCRASENIRSNLAWYQQKPGKSPKLLVNV
ATNLADGVPSRFSGSGSGTDYSLKINSLQPEDFGTYYCQHYWGTTWTFGQ
GTKLEIKR.

While the cell-binding agent preferably is an antibody, the cell-binding agent also can be a non-antibody molecule. Suitable non-antibody molecules include, for example, interferons (e.g., alpha, beta, or gamma interferon), lymphokines (e.g., interleukin 2 (IL-2), IL-3, IL-4, or IL-6), hormones (e.g., insulin), growth factors (e.g., EGF, TGF-alpha, FGF, and VEGF), colony-stimulating factors (e.g., G-CSF, M-CSF, and GM-CSF (see, e.g., Burgess, Immunology Today, 5: 155-158 (1984)), somatostatin, and transferrin (see, e.g., O'Keefe et al., J. Biol. Chem., 260: 932-937 (1985)). For example, GM-CSF, which binds to myeloid cells, can be used as a cell-binding agent to target acute myelogenous leukemia cells. In addition, IL-2, which binds to activated T-cells, can be used for prevention of transplant graft rejection, for therapy and prevention of graft-versus-host disease, and for treatment of acute T-cell leukemia. Epidermal growth factor (EGF) can be used to target squamous cancers such as lung cancer and head and neck cancer. Somatostatin can be used to target neuroblastoma cells and other tumor cell types.

The conjugate can comprise any suitable cytotoxic agent. A “cytotoxic agent,” as used herein, refers to any compound that results in the death of a cell, induces cell death, or decreases cell viability. Suitable cytotoxic agents include, for example, maytansinoids and conjugatable ansamitocins (see, for example, International Patent Application Publication WO 2012/061590 filed Nov. 3, 2011), taxoids, CC-1065 and CC-1065 analogs, and dolastatin and dolastatin analogs. In a preferred embodiment of the invention, the cytotoxic agent is a maytansinoid, including maytansinol and maytansinol analogs. Maytansinoids are compounds that inhibit microtubule formation and are highly toxic to mammalian cells. Examples of suitable maytansinol analogues include those having a modified aromatic ring and those having modifications at other positions. Such maytansinoids are described in, for example, U.S. Pat. Nos. 4,256,746, 4,294,757, 4,307,016, 4,313,946, 4,315,929, 4,322,348, 4,331,598, 4,361,650, 4,362,663, 4,364,866, 4,424,219, 4,371,533, 4,450,254, 5,475,092, 5,585,499, 5,846,545, and 6,333,410.

Examples of maytansinol analogs having a modified aromatic ring include: (1) C-19-dechloro (U.S. Pat. No. 4,256,746) (prepared by LAH reduction of ansamytocin P2), (2) C-20-hydroxy (or C-20-demethyl) +/−C-19-dechloro (U.S. Pat. Nos. 4,361,650 and 4,307,016) (prepared by demethylation using Streptomyces or Actinomyces or dechlorination using LAH), and (3) C-20-demethoxy, C-20-acyloxy (—OCOR), +/−dechloro (U.S. Pat. No. 4,294,757) (prepared by acylation using acyl chlorides).

Examples of maytansinol analogs having modifications of positions other than an aromatic ring include: (1) C-9-SH (U.S. Pat. No. 4,424,219) (prepared by the reaction of maytansinol with H₂S or P₂S₅), (2) C-14-alkoxymethyl (demethoxy/CH₂OR) (U.S. Pat. No. 4,331,598), (3) C-14-hydroxymethyl or acyloxymethyl (CH₂OH or CH₂OAc) (U.S. Pat. No. 4,450,254) (prepared from Nocardia), (4) C-15-hydroxy/acyloxy (U.S. Pat. No. 4,364,866) (prepared by the conversion of maytansinol by Streptomyces), (5) C-15-methoxy (U.S. Pat. Nos. 4,313,946 and 4,315,929) (isolated from Trewia nudiflora), (6) C-18-N-demethyl (U.S. Pat. Nos. 4,362,663 and 4,322,348) (prepared by the demethylation of maytansinol by Streptomyces), and (7) 4,5-deoxy (U.S. Pat. No. 4,371,533) (prepared by the titanium trichloride/LAH reduction of maytansinol).

In a preferred embodiment of the invention, the conjugate utilizes the thiol-containing maytansinoid DM1, also known as N^{2′}-deacetyl-N^{2′}-(3-mercapto-1-oxopropyl)-maytansine, as the cytotoxic agent. The structure of DM1 is represented by formula (I):

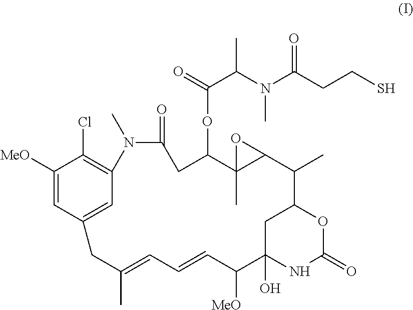 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In another preferred embodiment of the invention, the conjugate utilizes the thiol-containing maytansinoid DM4, also known as N^{2′}-deacetyl-N^{2′}-(4-methyl-4-mercapto-1-oxopentyl)-maytansine, as the cytotoxic agent. The structure of DM4 is represented by formula (II):

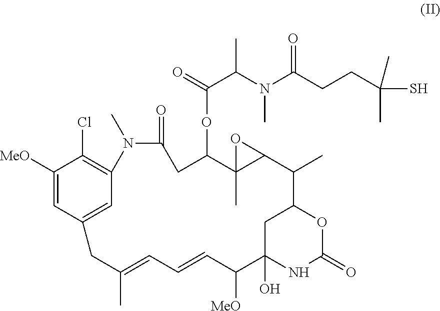 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

Other maytansinoids may be used in the context of the invention, including, for example, thiol and disulfide-containing maytansinoids bearing a mono or di-alkyl substitution on the carbon atom bearing the sulfur atom. Particularly preferred is a maytansinoid having at the C-3 position (a) C-14 hydroxymethyl, C-15 hydroxy, or C-20 desmethyl functionality, and (b) an acylated amino acid side chain with an acyl group bearing a hindered sulfhydryl group, wherein the carbon atom of the acyl group bearing the thiol functionality has one or two substituents, said substituents being CH₃, C₂H₅, linear or branched alkyl or alkenyl having from 1 to 10 carbon atoms, cyclic alkyl or alkenyl having from 3 to 10 carbon atoms, phenyl, substituted phenyl, or heterocyclic aromatic or heterocycloalkyl radical, and further wherein one of the substituents can be H, and wherein the acyl group has a linear chain length of at least three carbon atoms between the carbonyl functionality and the sulfur atom.

Additional maytansinoids for use in the context of the invention include compounds represented by formula (III):

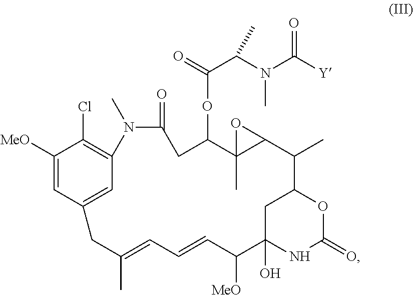

wherein Y′ represents
(CR₇R₈)₁(CR₉═CR₁₀)ₚ(C≡C)Aₒ(CR₅R₆)ₘDᵤ(CR₁₁═CR₁₂)ᵣ(C≡C)ₛBₜ(CR₃R₄)ₙCR₁R₂SZ, wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein A, B, D are cycloalkyl or cycloalkenyl having 3-10 carbon atoms, simple or substituted aryl, or heterocyclic aromatic, or heterocycloalkyl radical,
wherein R₃, R₄, R₅, R₆, R₇, R₈, R₉, R₁₀, R₁₁, and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic, or heterocycloalkyl radical,
wherein l, m, n, o, p, q, r, s, and t are each independently zero or an integer from 1 to 5, provided that at least two of l, m, n, o, p, q, r, s and t are not zero at any one time, and wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic, or heterocycloalkyl radical.

Preferred embodiments of formula (III) include compounds of formula (III) wherein (a) R₁ is H, R₂ is methyl and Z is H, (b) R₁ and R₂ are methyl and Z is H, (c) R₁ is H, R₂ is methyl, and Z is —SCH₃, and (d) R₁ and R₂ are methyl, and Z is —SCH₃.

Such additional maytansinoids also include compounds represented by formula (IV-L), (IV-D), or (IV-D,L):

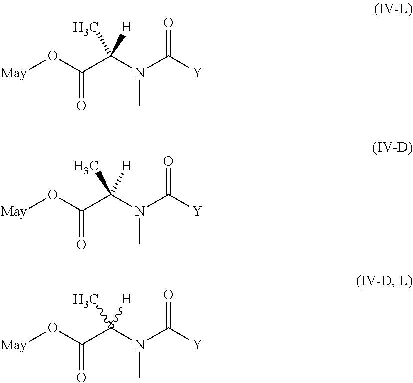

wherein Y represents (CR₇R₈)₁(CR₅R₆)ₘ(CR₃R₄)CR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero,
wherein Z is H, SR, or COR wherein R is linear or branched alkyl or alkenyl having from 1 to 10 carbon atoms, cyclic alkyl or alkenyl having from 3 to 10 carbon atoms, or simple or substituted aryl or heterocyclic aromatic or heterocycloalkyl radical, and
wherein May represents a maytansinoid which bears the side chain at C-3, C-14 hydroxymethyl, C-15 hydroxy, or C-20 desmethyl.

Preferred embodiments of formulas (IV-L), (IV-D) and (IV-D,L) include compounds of formulas (IV-L), (IV-D) and (IV-D,L) wherein (a) R₁ is H, R₂ is methyl, R₅, R₆, R₇, and R₈ are each H, l and m are each 1, n is 0, and Z is H, (b) R₁ and R₂ are methyl, R₅, R₆, R₇, R₈ are each H, l and m are 1, n is 0, and Z is H, (c) R₁ is H, R₂ is methyl, R₅, R₆, R₇, and R₈ are each H, l and m are each 1, n is 0, and Z is —SCH₃, or (d) R₁ and R₂ are methyl, R₅, R₆, R₇, R₈ are each H, l and m are 1, n is 0, and Z is SCH₃.

Preferably the cytotoxic agent is represented by formula (IV-L).

Additional preferred maytansinoids also include compounds represented by formula (V):

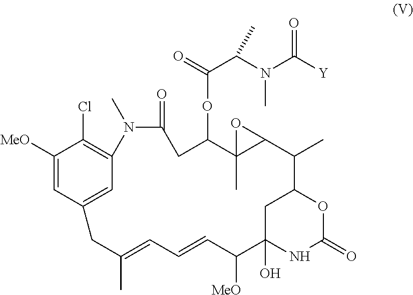

wherein Y represents (CR₇R₈)₁(CR₅R₆)ₘ(CR₃R₄)ₙCR₁R₂SZ,
wherein R₁ and R₂ are each independently CH₃, C₂H₅, linear alkyl, or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl, or heterocyclic aromatic or heterocycloalkyl radical,
wherein l, m, and n are each independently an integer of from 1 to 5, and in addition n can be zero, and
wherein Z is H, SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (V) include compounds of formula (V) wherein (a) R₁ is H, R₂ is methyl, R₅, R₆, R₇, and R₈ are each H; l and m are each 1; n is 0; and Z is H, (b) R₁ and R₂ are methyl; R₅, R₆, R₇, R₈ are each H, l and m are 1; n is 0; and Z is H, (c) R₁ is H, R₂ is methyl, R₅, R₆, R₇, and R₈ are each H, l and m are each 1, n is 0, and Z is —SCH₃, or (d) R₁ and R₂ are methyl, R₅, R₆, R₇, R₈ are each H, l and m are 1, n is 0, and Z is —SCH₃.

Still further preferred maytansinoids include compounds represented by formula (VI-L), (VI-D), or (VI-D,L):

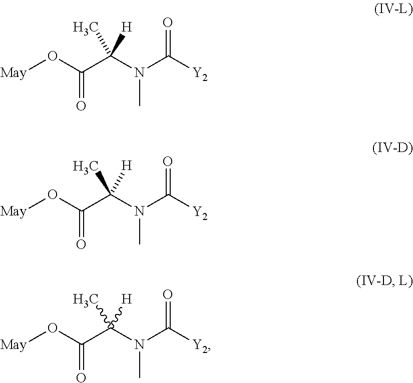

wherein Y₂ represents (CR₇R₈)₁(CR₅R₆)ₘ(CR₃R₄)ₙCR₁R₂SZ₂,
wherein R₁ and R₂ are each independently CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical, and wherein R₂ also can be H,
wherein R₃, R₄, R₅, R₆, R₇, and R₈ are each independently H, CH₃, C₂H₅, linear cyclic alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical,
wherein l, m, and n are each independently an integer of from 1 to 5, and in addition n can be zero,
wherein Z₂ is SR or COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical, and wherein May is the macrocyclic ring structure of the maytansinoid.

Additional preferred maytansinoids include compounds represented by formula (VII):

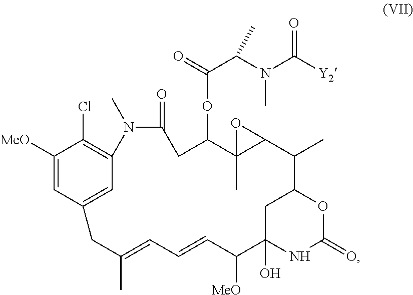

wherein Y_{2′} represents
(CR₇R₈)₁(CR₉═CR₁₀)ₚ(C≡C)_{q}Aₒ(CR₅R₆)ₘDᵤ(CR₁₁═CR₁₂)ᵣ(C≡C)ₛBₜ(CR₃R₄)ₙCR₁R₂SZ₂, wherein R₁ and R₂ are each independently CH₃, C₂H₅, linear branched or alkyl or alkenyl having from 1 to 10 carbon atoms, cyclic alkyl or alkenyl having from 3 to 10 carbon atoms, phenyl, substituted phenyl or heterocyclic aromatic or heterocycloalkyl radical, and in addition R₂ can be H,
wherein A, B, and D each independently is cycloalkyl or cycloalkenyl having 3 to 10 carbon atoms, simple or substituted aryl, or heterocyclic aromatic or heterocycloalkyl radical, wherein R₃, R₄, R₅, R₆, R₇, R₈, R₉, R₁₀, R₁₁, and R₁₂ are each independently H, CH₃, C₂H₅, linear alkyl or alkenyl having from 1 to 10 carbon atoms, branched or cyclic alkyl or alkenyl having from 3 to 10 carbon atoms, phenyl, substituted phenyl or heterocyclic aromatic or heterocycloalkyl radical,
wherein l, m, n, o, p, q, r, s, and t are each independently zero or an integer of from 1 to 5, provided that at least two of l, m, n, o, p, q, r, s and t are not zero at any one time, and wherein Z₂ is SR or —COR, wherein R is linear alkyl or alkenyl having from 1 to 10 carbon atoms, branched or cyclic alkyl or alkenyl having from 3 to 10 carbon atoms, or simple or substituted aryl or heterocyclic aromatic or heterocycloalkyl radical.

Preferred embodiments of formula (VII) include compounds of formula (VII), wherein R₁ is H and R₂ is methyl.

In addition to maytansinoids, the cytotoxic agent used in the conjugate can be a taxane or derivative thereof. Taxanes are a family of compounds that includes paclitaxel (Taxol®), a cytotoxic natural product, and docetaxel (Taxotere®), a semi-synthetic derivative, which are both widely used in the treatment of cancer. Taxanes are mitotic spindle poisons that inhibit the depolymerization of tubulin, resulting in cell death. While docetaxel and paclitaxel are useful agents in the treatment of cancer, their antitumor activity is limited because of their non-specific toxicity towards normal cells. Further, compounds like paclitaxel and docetaxel themselves are not sufficiently potent to be used in conjugates of cell-binding agents.

A preferred taxane for use in the preparation of a cytotoxic conjugate is the taxane of formula (VIII):

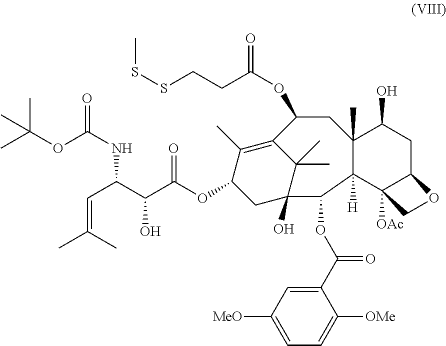 [SMILES: COc1ccc(OC)c(C(=O)O[C@H]2[C@H]3[C@](C)(C[C@H](OC(=O)CCSSC)C4=C(C)[C@@H](OC(=O)[C@H](O)[C@H](C=C(C)C)NC(=O)OC(C)(C)C)C[C@]2(O)C4(C)C)[C@@H](O)C[C@H]2OC[C@]23OC(C)=O)c1]

Methods for synthesizing taxanes that can be used in the context of the invention, along with methods for conjugating taxanes to cell-binding agents such as antibodies, are described in detail in U.S. Pat. Nos. 5,416,064, 5,475,092, 6,340,701, 6,372,738, 6,436,931, 6,596,757, 6,706,708, 6,716,821, and 7,390,898.

The cytotoxic agent also can be CC-1065 or a derivative thereof. CC-1065 is a potent anti-tumor antibiotic isolated from the culture broth of Streptomyces zelensis. CC-1065 is about 1000-fold more potent in vitro than commonly used anti-cancer drugs, such as doxorubicin, methotrexate, and vincristine (Bhuyan et al., Cancer Res., 42: 3532-3537 (1982)). CC-1065 and its analogs are disclosed in U.S. Pat. Nos. 5,585,499, 5,846,545, 6,340,701, and 6,372,738. The cytotoxic potency of CC-1065 has been correlated with its alkylating activity and its DNA-binding or DNA-intercalating activity. These two activities reside in separate parts of the molecule. In this respect, the alkylating activity is contained in the cyclopropapyrroloindole (CPI) subunit and the DNA-binding activity resides in the two pyrroloindole subunits of CC-1065.

Several CC-1065 analogs are known in the art and also can be used as the cytotoxic agent in the conjugate (see, e.g., Warpehoski et al., J. Med. Chem., 31: 590-603 (1988)). A series of CC-1065 analogs has been developed in which the CPI moiety is replaced by a cyclopropabenzindole (CBI) moiety (Boger et al., J. Org. Chem., 55: 5823-5833 (1990), and Boger et al., Bioorg. Med. Chem. Lett., 1: 115-120 (1991)). These CC-1065 analogs maintain the high in vitro potency of the parental drug, without causing delayed toxicity in mice. Like CC-1065, these compounds are alkylating agents that covalently bind to the minor groove of DNA to cause cell death.

The therapeutic efficacy of CC-1065 analogs can be greatly improved by changing the in vivo distribution through targeted delivery to a tumor site, resulting in lower toxicity to non-targeted tissues, and thus, lower systemic toxicity. To this end, conjugates of analogs and derivatives of CC-1065 with cell-binding agents that specifically target tumor cells have been generated (see, e.g., U.S. Pat. Nos. 5,475,092, 5,585,499, and 5,846,545). These conjugates typically display high target-specific cytotoxicity in vitro, and anti-tumor activity in human tumor xenograft models in mice (see, e.g., Chari et al., Cancer Res., 55: 4079-4084 (1995)).

Methods for synthesizing CC-1065 analogs are described in detail in U.S. Pat. Nos. 5,475,092, 5,585,499, 5,846,545, 6,534,660, 6,586,618, 6,756,397, and 7,329,760.

Drugs such as methotrexate, daunorubicin, doxorubicin, vincristine, vinblastine, melphalan, mitomycin C, chlorambucil, calicheamicin, tubulysin and tubulysin analogs, duocarmycin and duocarmycin analogs, dolastatin and dolastatin analogs also can be used as the cytotoxic agents of the invention. Doxarubicin and daunorubicin compounds (see, e.g., U.S. Pat. No. 6,630,579) can also be used as the cytotoxic agent.

The cell-binding agent cytotoxic agent conjugates may be prepared by in vitro methods. In order to link a cytotoxic agent to the antibody, a linking group is used. Suitable linking groups are well known in the art and include disulfide groups, acid labile groups, photolabile groups, peptidase labile groups, and esterase labile groups, as well as noncleavable linking groups.

In accordance with the invention, the cell-binding agent is modified by reacting a bifunctional crosslinking reagent with the cell-binding agent, thereby resulting in the covalent attachment of a linker molecule to the cell-binding agent. As used herein, a “bifunctional crosslinking reagent” refers to a reagent that possesses two reactive groups; one of which is capable of reacting with a cell-binding agent, while the other one is capable of reacting with the cytotoxic agent to link the cell-binding agent with the cytotoxic agent, thereby forming a conjugate.

Any suitable bifunctional crosslinking reagent can be used in connection with the invention, so long as the linker reagent provides for retention of the therapeutic, e.g., cytotoxicity, and targeting characteristics of the cytotoxic agent and the cell-binding agent, respectively, while providing an acceptable toxicity profile. Preferably, the linker molecule joins the cytotoxic agent to the cell-binding agent through chemical bonds (as described above), such that the cytotoxic agent and the cell-binding agent are chemically coupled (e.g., covalently bonded) to each other.

In one embodiment, the bifunctional crosslinking reagent comprises non-cleavable linkers. A non-cleavable linker is any chemical moiety that is capable of linking a cytotoxic agent, such as a maytansinoid, a taxane, or a CC-1065 analog, to a cell-binding agent in a stable, covalent manner. Thus, non-cleavable linkers are substantially resistant to acid-induced cleavage, light-induced cleavage, peptidase-induced cleavage, esterase-induced cleavage, and disulfide bond cleavage, at conditions under which the cytotoxic agent or the cell-binding agent remains active.

Suitable crosslinking reagents that form non-cleavable linkers between a cytotoxic agent and the cell-binding agent are well known in the art. In one embodiment, the cytotoxic agent is linked to the cell-binding agent through a thioether bond. In another embodiment, the cytotoxic agent is linked to the cell-binding agent through an amide bond. Examples of non-cleavable linkers include linkers having a maleimido- or haloacetyl-based moiety for reaction with the cytotoxic agent. Such bifunctional crosslinking agents are well known in the art (see U.S. Patent Application Publication Nos. 2010/0129314, 2009/0274713, 2008/0050310, 2005/0169933, and Pierce Biotechnology Inc. P.O. Box 117, Rockland, Ill. 61105, USA) and include, but not limited to, N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), K-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), N-succinimidyl 3-(bromoacetamido)propionate (SBAP), bis-maleimidopolyethyleneglycol (BMPEO), BM(PEO)₂, BM(PEO)₃, N-(β-maleimidopropyloxy)succinimide ester (BMPS), 5-maleimidovaleric acid NHS, HBVS, 4-(4-N-maleimidophenyl)-butyric acid hydrazide.HCl (MPBH), Succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-Maleimidobenzoyl-N-hydroxysulfosuccinimide ester (sulfo-MBS), N-(γ-maleimidobutryloxy)sulfosuccinimde ester (sulfo-GMBS), N-(ε-maleimidocaproyloxy)sulfosuccimido ester (sulfo-EMCS), N-(κ-maleimidoundecanoyloxy)sulfosuccinimide ester (sulfo-KMUS), sulfosuccinimidyl 4-(p-maleimidophenyl)butyrate (sulfo-SMPB), CX1-1, sulfo-Mal, and PEG-Mal. Preferably, the bifunctional crosslinking reagent is SMCC.

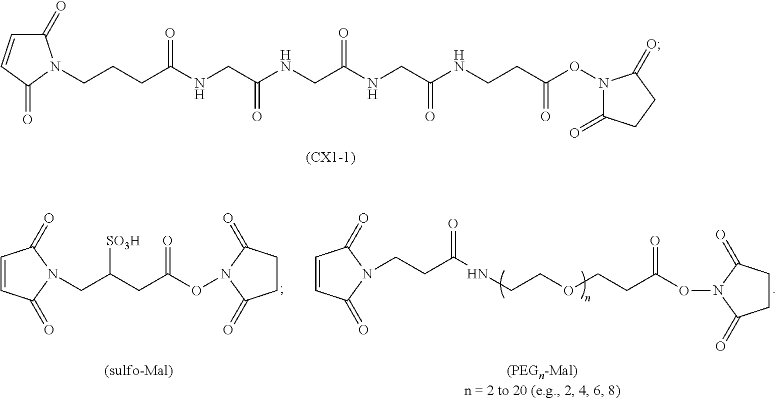

In one embodiment, the linking reagent is a cleavable linker. Examples of suitable cleavable linkers include disulfide containing linkers, acid labile linkers, photolabile linkers, peptidase labile linkers, and esterase labile linkers. Disulfide containing linkers are linkers cleavable through disulfide exchange, which can occur under physiological conditions. Acid labile linkers are linkers cleavable at acid pH. For example, certain intracellular compartments, such as endosomes and lysosomes, have an acidic pH (pH 4-5), and provide conditions suitable to cleave acid labile linkers. Photo labile linkers are useful at the body surface and in many body cavities that are accessible to light. Furthermore, infrared light can penetrate tissue. Peptidase labile linkers can be used to cleave certain peptides inside or outside cells (see e.g., Trouet et al., Proc. Natl. Acad. Sci. USA, 79: 626-629 (1982), and Umemoto et al., Int. J. Cancer, 43: 677-684 (1989)). In one embodiment, the cleavable linker is cleaved under mild conditions, i.e., conditions within a cell under which the activity of the cytotoxic agent is not affected.

In one embodiment, the cytotoxic agent is linked to a cell-binding agent through a disulfide bond. The linker molecule comprises a reactive chemical group that can react with the cell-binding agent. Preferred reactive chemical groups for reaction with the cell-binding agent are N-succinimidyl esters and N-sulfosuccinimidyl esters. Additionally the linker molecule comprises a reactive chemical group, preferably a dithiopyridyl group, that can react with the cytotoxic agent to form a disulfide bond. Bifunctional crosslinking reagents that enable the linkage of the cell-binding agent with the cytotoxic agent via disulfide bonds are known in the art and include, for example, N-succinimidyl 3-(2-pyridyldithio)propionate (SPDP) (see, e.g., Carlsson et al., Biochem. J., 173: 723-737 (1978)), N-succinimidyl 4-(2-pyridyldithio)butanoate (SPDB) (see, e.g., U.S. Pat. No. 4,563,304), N-succinimidyl 4-(2-pyridyldithio)pentanoate (SPP) (see, e.g., CAS Registry number 341498-08-6), and N-succinimidyl-4-(2-pyridyldithio)2-sulfo butanoate (sulfo-SPDB) (see, e.g., U.S. Patent Application Publication No. 2009/0274713). Other bifunctional crosslinking reagents that can be used to introduce disulfide groups are known in the art and are described in U.S. Pat. Nos. 6,913,748, 6,716,821 and U.S. Patent Application Publications 2009/0274713 and 2010/0129314, all of which are incorporated herein in its entirety by reference.

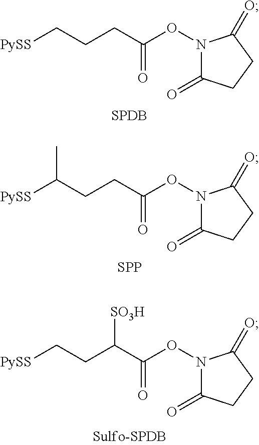

Other crosslinking reagents lacking a sulfur atom that form non-cleavable linkers can also be used in the inventive method. Such linkers can be derived from dicarboxylic acid based moieties. Suitable dicarboxylic acid based moieties include, but are not limited to, α,ω-dicarboxylic acids of the general formula (IX):

HOOC—X₁—Yₙ—Zₘ—COOH (IX),

wherein X is a linear or branched alkyl, alkenyl, or alkynyl group having 2 to 20 carbon atoms, Y is a cycloalkyl or cycloalkenyl group bearing 3 to 10 carbon atoms, Z is a substituted or unsubstituted aromatic group bearing 6 to 10 carbon atoms, or a substituted or unsubstituted heterocyclic group wherein the hetero atom is selected from N, O or S, and wherein l, m, and n are each 0 or 1, provided that l, m, and n are all not zero at the same time.

Many of the non-cleavable linkers disclosed herein are described in detail in U.S. Patent Application Publication No. 2005/0169933 A1.

Final purified cell-binding agent cytotoxic agent conjugates produced by the inventive process comprise a cytotoxic agent, a bifunctional crosslinking agent, and a cell-binding agent. In a preferred embodiment, the cytotoxic agent is DM4 and the bifunctional crosslinking agent is SPDB and the cell-binding agent is huB4 antibody. In another preferred embodiment, the cytotoxic agent is DM4 and the bifunctional crosslinking agent is SPDB and the cell-binding agent is huDS6 antibody. In another preferred embodiment, the cytotoxic agent is DM4 and the bifunctional crosslinking agent is SPDB and the cell-binding agent is B-B4 antibody. In another preferred embodiment, the cytotoxic agent is DM4 and the bifunctional crosslinking agent is sulfo-SPDB and the cell-binding agent is huMov19.

In yet another preferred embodiment of the invention, the cytotoxic agent is DM1, the bifunctional crosslinking agent is SMCC, and the cell-binding agent is huCD37-3 antibody. In another preferred embodiment of the invention, the cytotoxic agent is DM1, the bifunctional crosslinking agent is SMCC, and the cell-binding agent is EGFR-7R antibody. In a preferred embodiment of the invention, the cytotoxic agent is DM1, the bifunctional crosslinking agent is SMCC, and the cell-binding agent is an anti-EFGRvIII antibody. In a preferred embodiment of the invention, the cytotoxic agent is DM1, the bifunctional crosslinking agent is SMCC, and the cell-binding agent is an anti-CD27L antibody. In a preferred embodiment of the invention, the cytotoxic agent is DM1, the bifunctional crosslinking agent is SMCC, and the cell-binding agent is trastuzumab.

The following examples further illustrate the invention but, of course, should not be construed as in any way limiting its scope.

### Example 1

This example demonstrates the beneficial effect of performing the modification reaction of a process for preparing a cell-binding agent-cytotoxic agent conjugate at a high pH. In particular, this example demonstrates that contacting a cell-binding agent with a bifunctional crosslinking reagent in a solution having a pH greater than 7.0 has a beneficial effect on the stability of an antibody-maytansinoid conjugate.

Humanized N901 (huN901) antibody was reacted with heterobifunctional crosslinking reagent SPP in solutions of pH 7.0, pH 7.5, pH 7.8, and pH 8.0 to form a modified antibody. Specifically, 16 mg/ml of huN901 was reacted with the SPP at a ratio of 12.1 mg/g Ab (5.2X molar ratio) in 50 mM KPi buffer with 2 mM EDTA, 50 mM KCl and 5% (v/v) DMA at pH 7.0, pH 7.5, pH 7.8, and pH 8.0 for a total of 180 minutes at 20° C. with constant shaking. Then the samples were diluted and the pH was adjusted by adding H₂O, 250 mM EDTA at pH 8.0, and 0.5 M Citric Acid, to 2.6 mg/ml of Ab, 8.2 mM KPi, 8.2 mM KCl, and 2 mM EDTA, at pH 5.0 and held for 24 hours at room temperature.

The samples were conjugated at 2.5 mg/ml with 2.6 fold molar excess of DM1 relative to the initial SPP linker in 7.8 mM KPi buffer with 7.8 mM KCl, 2 mM EDTA, and 5.8% (v/v) DMA at pH 5.0 for 20 hours at room temperature with constant stirring. All the samples were purified by G25 NAP columns into 10 mM Succinate buffer with 250 mM Glycine at pH 5.5

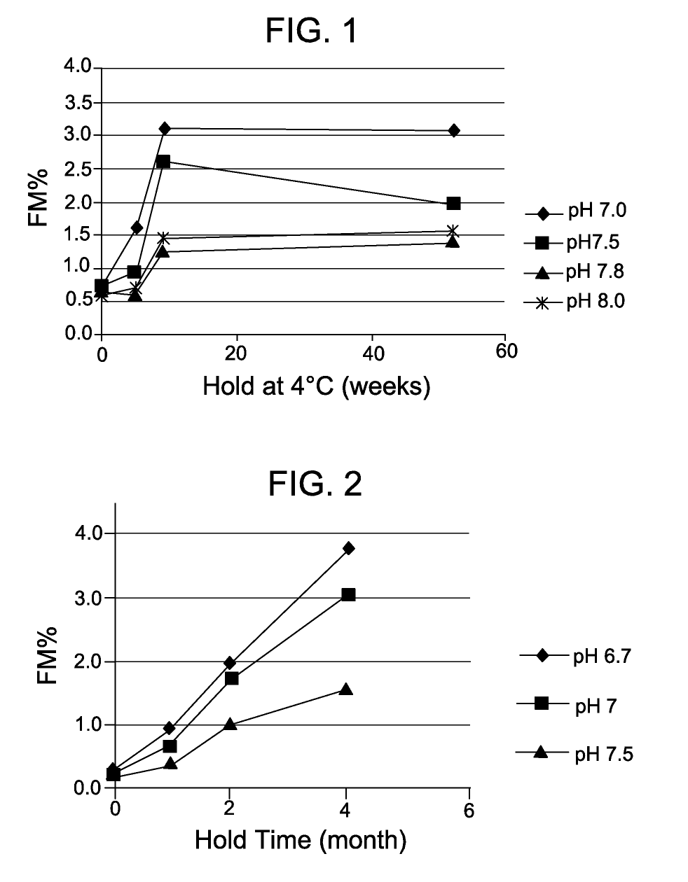

The conjugate samples were formulated to 1.0 mg/ml in formulation buffer containing 10 mM succinate, 250 mM glycine, 0.5% sucrose, and 0.01% Tween-20 at pH 5.5 and then put on a stability test at a storage temperature of 4° C. for 48 weeks. The stability of each sample was measured by analyzing the percentage of free maytansinoid (FM %) in each sample by HPLC over time, wherein formulations having the lowest percentage of free maytansinoid release over time are considered to be the most stable. As shown in FIG. 1, the three samples that were prepared by performing the modification reaction at a pH above 7.0 (i.e., pH 7.5, pH 7.8, and pH 8.0) exhibited less free maytansinoid release (FM %) over time than the sample that was prepared by performing the modification reaction at a pH of 7.0.

The results of the experiments reflected in this example demonstrate that cell-binding agent-cytotoxic agent conjugates prepared by performing the modification reaction at a higher pH are more stable than those from modification at lower pH upon storage (i.e., less release of free maytansinoid species). In particular, these results demonstrate that contacting a cell-binding agent with a bifunctional crosslinking reagent in a solution having a pH of 7.5, 7.8, or 8.0 has a beneficial effect on the stability of an antibody-maytansinoid conjugate.

### Example 2

This example demonstrates the beneficial effect of performing the modification reaction of a process for preparing a cell-binding agent-cytotoxic agent conjugate at a high pH. In particular, this example demonstrates that contacting a cell-binding agent with a bifunctional crosslinking reagent in a solution having a pH greater than 7.0 has a beneficial effect on the stability of an antibody-maytansinoid conjugate.

HuN901 antibody was modified at 9 mg/ml with SMCC at a ratio of 18.0 mg/g Ab (5.2X molar ratio) in 50 mM KPi buffer with 2 mM EDTA, 50 mM KCl, and 5% (v/v) DMA at pH 6.7, pH 7.0, and pH 7.5 for a total of 180 minutes at 20° C. with constant shaking. Then the samples were purified by G25 NAP columns into 10 mM succinate buffer at pH 5.5. Linker to antibody ratios (LAR) were determined.

The samples were conjugated at 5.0 mg/ml with 1.3 fold molar excess of DM1 relative to the bound linker (LAR) in 10 mM succinate at pH 5.0 for 20 hours at room temperature with constant stirring. All the samples were purified by G25 NAP columns into 10 mM succinate buffer with 250 mM glycine at pH 5.5. The stability of each sample was measured by analyzing the percentage of free maytansinoid in each sample by HPLC over time at 4° C., wherein formulations having the lowest percentage of free maytansinoid release over time are considered to be the most stable. As shown in FIG. 2, the sample that was prepared by performing the modification reaction at a pH of 7.5 exhibited less free maytansinoid release (FM %) over time than the samples that were prepared by performing the modification reaction at a pH of 6.7 and 7.0.

The results of the experiments reflected in this example demonstrate that cell-binding agent-cytotoxic agent conjugates prepared by performing the modification reaction at a higher pH are more stable than those from modification at lower pH upon storage (i.e., less release of free maytansinoid species). In particular, these results demonstrate that contacting a cell-binding agent with a bifunctional crosslinking reagent in a solution having a pH of 7.5 has a beneficial effect on the stability of an antibody-maytansinoid conjugate.

### Example 3

This example demonstrates the beneficial effect of performing the modification reaction of a process for preparing a cell-binding agent-cytotoxic agent conjugate at a high pH. In particular, this example demonstrates that contacting a cell-binding agent with a bifunctional crosslinking reagent in a solution having a pH of 7.5 has a beneficial effect on the stability of an antibody-maytansinoid conjugate.

A humanized anti-CD37 antibody was modified at 8 mg/ml with SPDB in 50 mM KPi buffer with 2 mM EDTA and 5% (v/v) DMA at pH 6.5 at a molar ratio of 5.9X (linker/Ab) or at pH 7.5 at a molar ratio of 5.4X (linker/Ab) for a total of 180 minutes at 20° C. with constant shaking A humanized anti-CD33 antibody was modified at 8 mg/ml with SPDB in 50 mM KPi buffer with 2 mM EDTA and 5% (v/v) DMA at pH 6.5 at a molar ratio of 5.9X (linker/Ab) or at pH 7.5 at a molar ratio of 5.4X (linker/Ab) for a total of 180 minutes at 20° C. with constant shaking

Two different processes were then used to conjugate the modified antibodies to DM4. The first process comprises a single purification after the conjugation reaction (one purif.). The second process comprises a first purification step between the modification and conjugation reactions and a second purification step after the conjugation reaction (two purif.).

For the first process, samples from the pH 7.5 modification reactions described above were pH adjusted to pH 6.5 by adding 0.5 M citric Acid, and further diluted to 2.6 mg/ml of Ab, by using pH 6.5 buffer containing 50 mM KPi and 2 mM EDTA. The samples were then conjugated at 2.5 mg/ml with a molar ratio of 1.7X (DM4 relative to the initial SPDB) in pH 6.5 buffer with 5% (v/v) DMA for 20 hours at room temperature with constant stirring.

For the second process, samples from the modification reactions described above were purified by a NAP G25 column using a pH 6.5 buffer. Antibody concentration and linker to antibody ratio (LAR) were measured. The samples were then conjugated at 2.5 mg/ml with a molar ratio of 1.7X (DM4 relative to LAR) in pH 6.5 buffer with 5% (v/v) DMA for 20 hours at room temperature with constant stirring.

After the conjugation reaction, all the samples were purified by G25 NAP columns into 10 mM succinate, 250 mM glycine and pH 5.5 buffer. Conjugate MAR and free maytansinoid (FM) were measured. Then the conjugate was formulated at 1 g/L in buffer containing 10 mM succinate, 250 glycine, 0.5% sucrose, and 0.1% polysorbate 20. Samples of each antibody conjugate formulation were stored at 4° C., 25° C., or 40° C. for stability testing.

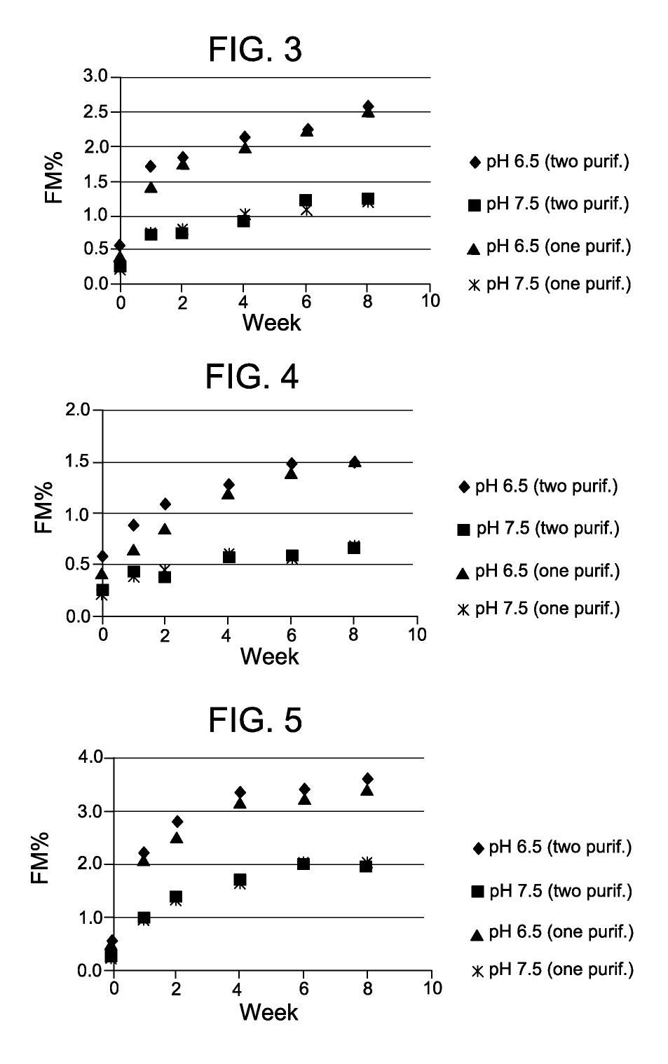

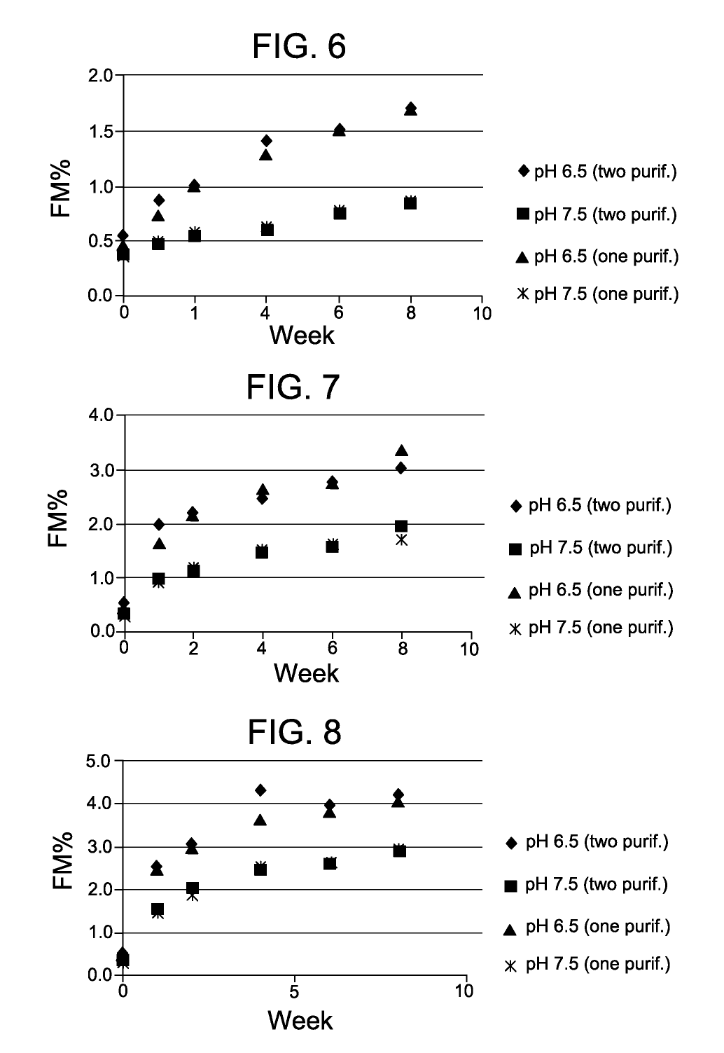

The stability of each sample was measured by analyzing the percentage of free maytansinoid in each sample by HPLC over time, wherein formulations having the lowest percentage of free maytansinoid release over time are considered to be the most stable. As shown in FIGS. 3-8, all of the samples that were prepared by performing the modification reaction at a pH of 7.5 exhibited less free maytansinoid release (FM %) over time than the samples that were prepared by performing the modification reaction at a pH of 6.5.

The results of the experiments reflected in this example demonstrate that two different cell-binding agent-cytotoxic agent conjugates prepared by two different processes in which the modification reaction was performed at a higher pH are more stable than those from modification at lower pH upon storage (i.e., less release of free maytansinoid species over time). In particular, these results demonstrate that contacting a cell-binding agent with a bifunctional crosslinking reagent in a solution having a pH of 7.5 has a beneficial effect on the stability of two different antibody-maytansinoid conjugates.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A process for preparing a cell-binding agent having a linker bound thereto, which process comprises contacting a cell-binding agent with a bifunctional crosslinking reagent in a solution having a pH of 7.5 to 9 to covalently attach a linker to the cell-binding agent and thereby prepare a mixture comprising cell-binding agents having linkers bound thereto.

2. The process of claim 1, wherein the pH is about 8.0.

3. The process of claim 1, wherein the pH is 7.8, 7.9. 8.0, 8.1 or 8.2.

4. The process of claim 1, wherein the solution comprises a buffering agent selected from a citrate buffer, an acetate buffer, a succinate buffer, and a phosphate buffer.

5. The process of claim 1, wherein the solution comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

6. The process of claim 1, wherein the contacting occurs at a temperature of about 16° C. to about 25° C.

7. The process of claim 6, wherein the temperature is about 20° C.

8. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent in a solution having a pH of 7.5 to 9 to covalently attach a linker to the cell-binding agent and thereby prepare a first mixture comprising cell-binding agents having linkers bound thereto,
(b) subjecting the first mixture to tangential flow filtration, selective precipitation, non-adsorptive chromatography, adsorptive filtration, adsorptive chromatography, or a combination thereof and thereby prepare a purified first mixture of cell-binding agents having linkers bound thereto,
(c) conjugating a cytotoxic agent to the cell-binding agents having linkers bound thereto in the purified first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and
(d) subjecting the second mixture to tangential flow filtration, selective precipitation, non-adsorptive chromatography, adsorptive filtration, adsorptive chromatography, or a combination thereof to purify the cell-binding agent chemically coupled through the linker to the cytotoxic agent from the other components of the second mixture and thereby prepare a purified second mixture of cell-binding agent chemically coupled through the linker to the cytotoxic agent.

9. The process of claim 8, wherein the contacting in step (a) occurs in a solution having a pH of about 8.0.

10. The process of claim 8, wherein the solution in step (a) comprises a buffering agent selected from the group consisting of a citrate buffer, an acetate buffer, a succinate buffer, and a phosphate buffer.

11. The process of claim 8, wherein the solution in step (a) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

12. The process of claim 8, wherein the contacting in step (a) occurs at a temperature of about 16° C. to about 25° C.

13. The process of claim 12, wherein the temperature is about 20° C.

14. The process of claim 8, wherein the non-adsorptive chromatography is selected from the group consisting of SEPHADEX™ resins, SEPHACRYL™ resins, SUPERDEX™ resins, and BIO-GEL® resins.

15. The process of claim 8, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

16. The process of claim 8, wherein tangential flow filtration is utilized in steps (b) and (d).

17. The process of claim 8, wherein adsorptive chromatography is utilized in steps (b) and (d).

18. The process of claim 8, wherein non-adsorptive chromatography is utilized in steps (b) and (d).

19. The process of claim 8, wherein tangential flow filtration is utilized in step (b) and adsorptive chromatography is utilized in step (d).

20. The process of claim 8, wherein adsorptive chromatography is utilized in step (b) and tangential flow filtration is utilized in step (d).

21. The process of claim 8, wherein the adsorptive chromatography is ion-exchange chromatography.

22. The process of claim 8, wherein the adsorptive chromatography is ceramic hydroxyapatite (CHT) chromatography.

23. The process of claim 8, wherein the solution in step (c) comprises sucrose.

24. The process of claim 8, wherein the solution in step (c) comprises a buffering agent selected from the group consisting of a citrate buffer, an acetate buffer, a succinate buffer, and a phosphate buffer.

25. The process of claim 8, wherein the solution in step (c) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

26. The process of claim 8, further comprising
(e) holding the mixture between at least one of steps a-b, steps b-c, and steps c-d to release the unstably bound linkers from the cell-binding agent.

27. A process for preparing a cell-binding agent-cytotoxic agent conjugate comprising a cell-binding agent chemically coupled to a cytotoxic agent through a linker, which process comprises:
(a) contacting a cell-binding agent with a bifunctional crosslinking reagent in a solution having a pH of 7.5 to 9 to covalently attach a linker to the cell-binding agent and thereby prepare a first mixture comprising cell-binding agents having linkers bound thereto,
(b) conjugating a cytotoxic agent to the cell-binding agents having linkers bound thereto in the first mixture by reacting the cell-binding agents having linkers bound thereto with a cytotoxic agent in a solution having a pH of about 4 to about 9 to prepare a second mixture comprising (i) cell-binding agent chemically coupled through the linker to the cytotoxic agent, (ii) free cytotoxic agent, and (iii) reaction by-products, and
(c) subjecting the second mixture to tangential flow filtration, selective precipitation, non-adsorptive chromatography, adsorptive filtration, adsorptive chromatography, or a combination thereof, to purify the cell binding agent chemically coupled through the linker to the cytotoxic agent from the other components of the second mixture and thereby prepare a purified second mixture of cell binding agent chemically coupled through the linker to the cytotoxic agent.

28. The process of claim 27, wherein the first mixture is not subjected to purification between steps (a) and (b).

29. The process of claim 27, wherein the contacting in step (a) occurs in a solution having a pH of about 8.0.

30. The process of claim 27, wherein the solution in step (a) comprises a buffering agent selected from a citrate buffer, an acetate buffer, a succinate buffer, and a phosphate buffer.

31. The process of claim 27, wherein the solution in step (a) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

32. The process of claim 27, wherein the contacting in step (a) occurs at a temperature of about 16° C. to about 25° C.

33. The process of claim 32, wherein the temperature is about 20° C.

34. The process of claim 27, wherein the non-adsorptive chromatography is selected from the group consisting of SEPHADEX™ resins, SEPHACRYL™ resins, SUPERDEX™ resins, and BIO-GEL® resins.

35. The process of claim 27, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

36. The process of claim 27, wherein the solution in step (b) comprises sucrose.

37. The process of claim 27, wherein the solution in step (b) comprises a buffering agent selected from the group consisting of a citrate buffer, an acetate buffer, a succinate buffer, and a phosphate buffer.

38. The process of claim 27, wherein the solution in step (b) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

39. The process of claim 27, further comprising
(d) holding the mixture between at least one of steps a-b and steps b-c to release the unstably bound linkers from the cell-binding agent.

40. A process for preparing a cell-binding agent-cytotoxic agent conjugate comprising a cell-binding agent chemically coupled to a cytotoxic agent through a linker, which process comprises:
(a) contacting a cell-binding agent with a cytotoxic agent-linker compound comprising a cytotoxic agent chemically coupled to a linker in a solution having a pH of 7.5 to 9 to covalently attach the cytotoxic agent-linker compound to the cell-binding agent and thereby prepare a mixture comprising the cell-binding agent-cytotoxic agent conjugate
(b) subjecting the mixture comprising the cell-binding agent-cytotoxic agent conjugate to tangential flow filtration, selective precipitation, non-adsorptive chromatography, adsorptive filtration, adsorptive chromatography or a combination thereof to purify the conjugate.

41. The process of claim 40, wherein the cytotoxic agent-linker compound is purified before contacting with the cell-binding agent.

42. The process of claim 40, wherein the cytotoxic agent-linker compound is not purified before contacting with the cell-binding agent.

43. The process of claim 40, wherein the cytotoxic agent-linker compound is prepared by contacting a cytotoxic agent with a bifunctional crosslinking reagent comprising a linker to covalently attach the cytotoxic agent to the linker and thereby prepare a mixture comprising the cytotoxic agent-linker compound.

44. The process of claim 40, wherein the contacting in step (a) occurs in a solution having a pH of about 8.0.

45. The process of claim 40, wherein the solution in step (a) comprises a buffering agent selected from a citrate buffer, an acetate buffer, a succinate buffer, and a phosphate buffer.

46. The process of claim 40, wherein the solution in step (a) comprises a buffering agent selected from the group consisting of HEPPSO (N-(2-Hydroxyethyl)piperazine-N′-(2-hydroxypropanesulfonic acid)), POPSO (Piperazine-1,4-bis-(2-hydroxy-propane-sulfonic acid) dehydrate), HEPES (4-(2-hydroxyethyl)piperazine-1-ethanesulfonic acid), HEPPS (EPPS) (4-(2-hydroxyethyl)piperazine-1-propanesulfonic acid), TES (N-[tris(hydroxymethyl)methyl]-2-aminoethanesulfonic acid), and a combination thereof.

47. The process of claim 40, wherein the contacting in step (a) occurs at a temperature of about 16° C. to about 25° C.

48. The process of claim 47, wherein the temperature is about 20° C.

49. The process of claim 40, wherein the non-adsorptive chromatography is selected from the group consisting of SEPHADEX™ resins, SEPHACRYL™ resins, SUPERDEX™ resins, and BIO-GEL® resins.

50. The process of claim 40, wherein the adsorptive chromatography is selected from the group consisting of hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

51. The process of claim 40, further comprising
(c) holding the mixture between steps a-b to release the unstably bound linkers from the cell-binding agent.

52. The process of claim 1, wherein the bifunctional crosslinking reagent is an acid labile linker, a photolabile linker, a peptidase labile linker, or an esterase labile linker.

53. The process of claim 1, wherein the bifunctional crosslinking reagent is a cleavable linker.

54. The process of claim 1, wherein the bifunctional crosslinking reagent is a disulfide containing linker.

55. The process of claim 1, wherein the bifunctional crosslinking reagent comprises an N-succinimidyl ester moiety, an N-sulfosuccinimidyl ester moiety, a maleimido-based moiety, or a haloacetyl-based moiety.

56. The process of claim 1, wherein the bifunctional crosslinking reagent is selected from the group consisting of N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), and N-succinimidyl-4-(2-pyridyldithio)2-sulfo butanoate (sulfo-SPDB).

57. The process of claim 1, wherein the bifunctional crosslinking reagent is selected from the group consisting of N-succinimidyl 4-(maleimidomethyl)cyclohexanecarboxylate (SMCC), N-succinimidyl-4-(N-maleimidomethyl)-cyclohexane-1-carboxy-(6-amidocaproate) (LC-SMCC), κ-maleimidoundecanoic acid N-succinimidyl ester (KMUA), γ-maleimidobutyric acid N-succinimidyl ester (GMBS), β-maleimidopropyloxy-succinimidyl ester (BMPS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), N-(p-maleimidophenyl)isocyanate (PMPI), sulfo-Mal, PEG₄-Mal, and CX1-1.

58. The process of claim 1, wherein the cell-binding agent is selected from the group consisting of antibodies, interferons, interleukin 2 (IL-2), interleukin 3 (IL-3), interleukin 4 (IL-4), interleukin 6 (IL-6), insulin, EGF, TGF-α, FGF, G-CSF, VEGF, MCSF, GM-CSF, and transferrin.

59. The process of claim 58, wherein the cell-binding agent is an antibody.

60. The process of claim 59, wherein the antibody is a monoclonal antibody.

61. The process of claim 60, wherein the antibody is a humanized monoclonal antibody.

62. The process of claim 1, wherein the cell-binding agent is an antibody selected from the group consisting of huB4, huC242, trastuzumab, bivatuzumab, sibrotuzumab, huDS6, rituximab, anti-CD33 antibody, anti-CD27L antibody, anti-Her2 antibody, anti-EGFR antibody, anti-EGFRvIII antibody, Cripto, anti-CD138 antibody, anti-CD38 antibody, anti-EphA2 antibody, integrin targeting antibody, anti-CD37 antibody, anti-folate receptor antibody, anti-Her3 antibody, B-B4 antibody, and anti-IGFIR antibody.

63. The process of claim 8, wherein the cytotoxic agent is selected from the group consisting of maytansinoids, taxanes, and CC1065.

64. The process of claim 8, wherein the cytotoxic agent is a maytansinoid.

65. The process of claim 64, wherein the maytansinoid comprises a thiol group.

66. The process of claim 65, wherein the maytansinoid is N^{2′}-deacetyl-N^{2′}-(3-mercapto-1-oxopropyl)-maytansine (DM1) or N^{2′}-de acetyl-N^{2′}-(4-methyl-4-mercapto-1-oxopentyl)-maytansine (DM4).

67. The process of claim 8, wherein the cytotoxic agent is DM4 and the bifunctional crosslinking agent is SPDB and the cell-binding agent is huB4 antibody.

68. The process of claim 8, wherein the cytotoxic agent is DM4 and the bifunctional crosslinking agent is SPDB and the cell-binding agent is huDS6 antibody.

69. The process of claim 8, wherein the cytotoxic agent is DM4 and the bifunctional crosslinking agent is SPDB and the cell-binding agent is B-B4 antibody.

70. The process of claim 8, wherein the cytotoxic agent is DM4 and the bifunctional crosslinking agent is sulfo-SPDB and the cell-binding agent is huMov19.

71. The process of claim 8, wherein the cytotoxic agent is DM1 and the bifunctional crosslinking agent is SMCC and the cell-binding agent is huCD37-3 antibody.

72. The process of claim 8, wherein the cytotoxic agent is DM1 and the bifunctional crosslinking agent is SMCC and the cell-binding agent is EGFR-7R antibody.

73. The process of claim 8, wherein the cytotoxic agent is DM1 and the bifunctional crosslinking agent is SMCC and the cell-binding agent is an anti-EFGRvIII antibody.

74. The process of claim 8, wherein the cytotoxic agent is DM1 and the bifunctional crosslinking agent is SMCC and the cell-binding agent is an anti-CD27L antibody.

75. The process of claim 8, wherein the cytotoxic agent is DM1 and the bifunctional crosslinking agent is SMCC and the cell-binding agent is trastuzumab.

76. The process of claim 8, wherein the cytotoxic agent is DM4 and the bifunctional crosslinking agent is SPDB and the cell-binding agent is Cripto.
